(19) United States
(12) Patent Application Publication
(10) Pub. No.: US 2024/0011975 A1
(43) Pub. Date: Jan. 11, 2024

(54) Nanopatterned Films with Patterned Surface Chemistry

(71) Applicant: 3M INNOVATIVE PROPERTIES COMPANY, St. Paul, MN (US)

(72) Inventors: Joshua M. Fishman, St. Louis Park, MN (US); Paul B. Armstrong, St. Paul, MN (US); Caleb T. Nelson, McKinney, TX (US); Kayla C. Niccum, Maplewood, MN (US); Henrik B. van Lengerich, St. Paul, MN (US); Tonya D. Bonilla, Woodbury, MN (US); Karl K. Stensvad, Eagan, MN (US)

(21) Appl. No.: 18/021,611

(22) PCT Filed: Sep. 8, 2021
(86) PCT No.: PCT/IB2021/058166

§ 371 Date: Feb. 16, 2023

Related U.S. Application Data
(60) Provisional application No. 63/078,850, filed on Sep. 15, 2020.

(51) Int. Cl. G01N 33/53; G01N 33/552; G01N 33/487

(52) U.S. Cl. CPC G01N 33/5302; G01N 33/552; G01N 33/487

(57) ABSTRACT

An article includes a flexible carrier film with a first major surface having an array of structures, at least a portion of which include an inorganic layer and an analyte binding layer. The analyte binding is bonded to the inorganic layer via a network of hydrocarbon linking groups, and the analyte binding layer includes at least one functional group selected to bind with a biochemical analyte. Recessed features interspersed with the structures are free of the inorganic layer and the analyte binding layer.

### BACKGROUND

The cost of high-throughput chemical and biological assays such as nucleic acid and peptide sequencing, or protein, gene and other biochemical and pharmaceutical assays, is driven by single-use consumables. For example, in addition to chemical and biological reagents used to probe, detect or measure the unknown sample, the high-throughput assays often require the use of a patterned flow cell consumable in which the chemical and biochemical reagents can selectively react with a target analyte.

Today, patterned flow cells in commercially available assays include glass or silicon substrates with etched nanowells. The nanowells in the glass substrates each include chemical functionality selected to bind a chosen target analyte and are separated by regions of an antifouling or non-interacting coatings or surface chemistry. In some examples, the chemical functionality can be a functionalized polymer or oligomer, for example a polyacrylamide containing hydrogel, or directly attached to the substrate via a small molecule linker. These patterned flow cells can be manufactured using an intricate and expensive wafer-based photolithographic process that includes multiple chemical-mechanical planarization (CMP), spin coating and washing steps to fill the nanowells with the correct chemical functionality, and place the anti-biofouling coating between the nanowells.

To reduce the financial burden for end users using high-throughput assays, and make the assays more accessible for new markets, it is necessary to reduce the costs of the consumables used in the analysis, including the glass substrates with etched nanowells.

### SUMMARY

In general, the present disclosure is directed to nanopatterned substrates for use in chemical or biological assays such as, for example, nucleic acid, protein, and other biochemical screening procedures, which are formed on a flexible carrier film. In various embodiments, the flexible carrier film can be structured with nanoscale posts or wells having functionalized analyte-binding regions selected to bind with a target analyte in an assay. In some embodiments, the functionalized posts or wells can be interspersed among anti-biofouling interstitial regions.

The nanopattemed flexible polymeric film substrates can be produced in a continuous manufacturing process, which can provide higher throughput and lower manufacturing costs compared to wafer-based photolithographic process methods that are generated on a parts basis. The nanopatterned flexible polymeric film substrates can be configured for use with a wide variety of assay reagents and instrumentation, and can be used in, for example, screening assays for genes or gene segments, single-nucleotide, polymorphism, RNA expression, non-coding RNAs, DNA methylation profiles, protein expression, peptides and other biochemical compounds, small molecules or biomarkers, as well as for chemical and environmental contaminants. In some embodiments, the flexible organic carrier film substrate can be adhesively mounted on a support layer, which can make it possible to use the substrate in screening instruments that currently employ more rigid silicon or glass substrate materials.

Continuous processing, which in some cases is also referred to as roll-to-roll processing, also provides several advantages and increased design flexibility relative to silicon wafer processing techniques when producing a nanostructured substrate. For example, when nanopatterned substrates are made using a silicon wafer, grafting analyte binding chemistry on post-like strictures extending away from the surface of the wafer can be difficult, and as a result in silicon wafer constructions the binding chemistry can be limited to the depressed well-like areas of the wafer. In addition, because thick inorganic layers take longer to deposit, and due to necessary flexibility of the polymer carrier web, the inorganic layer can be made thin (less than 200 nm). Further, in contrast to traditional wafer processing, amorphous silicon oxide layers deposited by roll-to-roll processing may include impurities such as aluminum or carbon to allow efficient deposition rates on flexible, temperature sensitive surfaces using processing techniques such as sputtering or plasma enhanced chemical vapor deposition (PECVD).

In one aspect, the present disclosure is directed to an article including a flexible carrier film with a first major surface and a second major surface, wherein a first major surface of the flexible carrier film includes comprises an array of structures extending away therefrom. At least a portion of the structures include an inorganic layer with a first major surface and a second major surface, wherein the first major surface of the inorganic layer is on the flexible carrier film, and an analyte binding layer with a first major surface on the second major surface of the inorganic layer, wherein the analyte binding is bonded to the inorganic layer via a network of hydrocarbon linking groups, and wherein the second major surface of the analyte binding layer includes at least one functional group selected to bind with a biochemical analyte. Recessed features are interspersed with the structures, wherein at least a portion of the recessed features are free of the inorganic layer and the analyte binding layer.

In another aspect, the present disclosure is directed to an article including a flexible carrier film with a first major surface and a second major surface; an inorganic layer with a first major surface and a second major surface, wherein the first major surface of the inorganic layer is on the first major surface of the flexible polymeric film; an anti-biofouling layer on at least a portion of the inorganic layer, wherein the anti-biofouling layer includes an arrangement of wells, wherein at least a portion of the wells comprise a floor having thereon a first major surface of an analyte binding layer bound to the second major surface of the inorganic layer via a network of hydrocarbon linking groups, and wherein a first major surface of the analyte binding layer in the well includes at least one functional group reactive with the analyte in the sample fluid; and structures interspersed with the wells, wherein at least a portion of the structures are free of the analyte binding layer and the inorganic layer.

In another aspect, the present disclosure is directed to a diagnostic device for detection of a biochemical analyte. The diagnostic device includes a flow cell with a patterned arrangement of fluidic channels configured to provide flow conduits for a sample fluid including the biochemical analyte, wherein at least some of the fluidic channels of the flow cell are lined on a surface thereof with: an arrangement of posts having an analyte binding layer on an exposed surface thereof, or an arrangement of wells comprising an analyte binding layer therein, wherein the analyte binding layer is configured to bind the biochemical analyte, and wherein the analyte binding layer is bonded to an underlying Si oxide layer by a network of methylene groups disposed on a flexible carrier film.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is schematic cross-sectional view of an embodiment of component article on a nanostructured flexible polymeric carrier substrate according to the present disclosure that includes functionalized posts.

FIG. 2 is schematic cross-sectional view of an embodiment of a component article on a nanostructured flexible polymeric carrier substrate according to the present disclosure that includes functionalized wells.

FIG. 3A is a schematic cross-sectional view of an example embodiment of a process for making the article of FIG. 1.

FIG. 3B is a schematic cross-sectional view of another example embodiment of a process for making the article of FIG. 1.

FIG. 4A is a schematic cross-sectional view of an embodiment of low-land transfer process utilized in the process of FIG. 3A.

FIG. 4B is a schematic cross-sectional view of an embodiment of low-land transfer process utilized in the process of FIG. 3B.

FIG. 5 is a schematic cross-sectional view of an example embodiment of a process for making the article of FIG. 2.

FIG. 6 is a schematic cross-sectional view of another example embodiment of a process for making the article of FIG. 2.

FIGS. 7A-7D are photographs of example component articles of FIG. 1 that include post constructions.

FIG. 8 is a plot of intensity vs wavelength of fluorescence response for various flexible carrier film substrates in the examples below.

FIG. 9A includes fluorescent images of various coatings of Group 1 after exposure to DNA in the plot of FIG. 10 in the examples below.

FIG. 9B includes fluorescent images of various coatings of Group 2 after exposure to DNA in the plot of FIG. 10 in the examples below.

FIG. 10 is a plot of fluorescence measurements of various substrates to quantify non-specific DNA absorption in the examples below.

Like symbols in the drawings indicate like elements.

### DETAILED DESCRIPTION

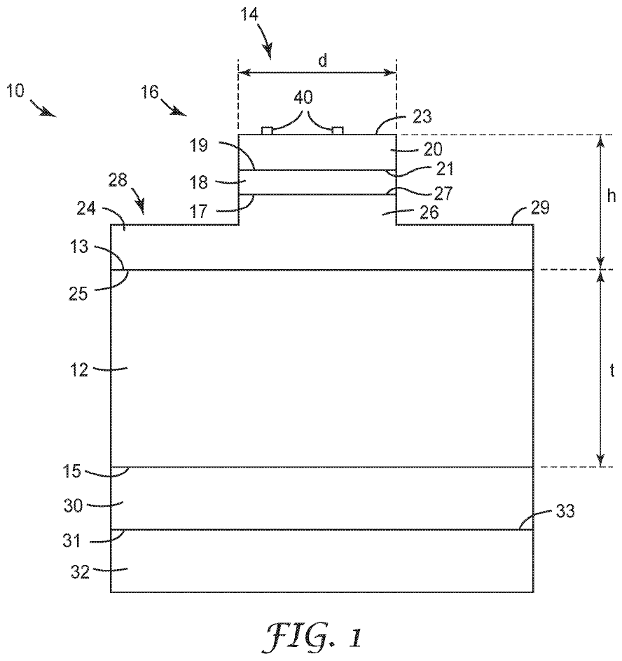

Referring now to FIG. 1, a schematic illustration (which is not to scale) of a portion of a component article 10 includes a flexible carrier film 12 substrate with a first major surface 13 and a second major surface 15. The flexible carrier film 12 may include any polymeric film suitable for use in a roll-to-roll process.

In some embodiments, the flexible carrier film 12 should be selected from a polymeric material with low autofluorescence to provide a low-noise background for biological assays in which, for example, fluorescent biological structures, fluorescent markers or fluorophores are used for analysis. For example, detection of DNA or RNA nucleotide sequences can be performed using fluorescent molecules. In other examples, which are not intended to be limiting, the fluorescent molecules can be labeled nucleotides, such as reversible terminators, or labeled oligonucleotide probes. In some instances, different labeled nucleotides or probes in a reagent kit are labeled with different fluorophores that emit different wavelengths depending on the specific sequence to enable multiple bases to be called in a single scan. An auto-fluorescent flexible carrier film 12 could potentially drown out the signal from these fluorescent sequencing reagents. In some examples, polymeric resins can optionally be modified to reduce fluorescence, which can make possible the use of a wider variety of polymeric materials for the flexible carrier film 12. In some examples, which are not intended to be limiting, the flexible carrier film 12 should have an autofluorescence measured between 400 nm and 800 nm, or between 450 nm and 650 nm, similar to that of borosilicate glass or other substrates commonly used in biological assays.

Autofluorescence is not a single number, as the spectrum emitted depends on the excitation wavelength, and a particular polymeric material can have high or low autofluorescence depending on the wavelength. In some examples, to provide low autofluoresence to detect a wide variety of biological detection molecules, cyclic olefin copolymers (COP) or biaxially oriented polypropylene (BOPP), which each have low autofluorescence across a wide spectrum, can be used. Other examples of suitable low autofluorescent polymeric films include, but are not limited to, poly(meth)acrylates and copolymers thereof, wherein (meth)acrylates include acrylates and methacrylates, polyamides, polyesters, polycarbonates such as, for example, those available under the trade designation Makrolon from Covestro AG, Pittsburgh, PA, hydrogenated styrenics such as, for example, cyclic block copolymers available from Vivion, Inc., San Carlos, CA, and mixtures and combinations thereof. In various embodiments, the flexible carrier film 12 can include a single or multiple layers of any of these polymers, and can have a total thickness t of about 5 μm to about 1000 μm.

At least a portion of the first major surface 13 of the flexible carrier film 12 includes an arrangement 14 of structures 16 extending away therefrom. In various embodiments, which are not intended to be limiting and provided as an example, the arrangement 14 may be a regular or an irregular array on the surface 13, and the structures 16 may be present in all or a portion of the surface 13. In the embodiment of FIG. 1, the structures 16 are generally cylindrical columns or posts, but the structures 16 may also have shapes such as spherical, pyramidal, cuboid, and the like. The structures 16 may include a wide variety of cross-sectional shapes such as, for example, substantially rectangular, arcuate, trapezoidal, cubic, and the like.

In some embodiments, the surface 13 of the flexible carrier film 12 may be structured by a wide variety of processes including, but not limited to, microreplication against a structured tool, casting, microcontact or inkjet printing, chemical treatment, laser patterning, and combinations thereof. In some embodiments, which are provided as an example, the arrangement of structures 14 includes a regular array of cylindrical or cuboid posts 16 with a diameter d of about 50 nm to about 10,000 nm, or about 200 nm and 7500 nm, and height h above the surface 13 of greater than 0 nm and up to about 1000 nm, or about 50 nm to about 200 nm. In some example embodiments, the posts have an aspect ratio (height:diameter) of about 5:1 to about 1:70, or about 5:1 to 1:5, or about 2:1 to 1:1. The array of posts 16 may occupy all or selected portions of the surface 13 of the flexible carrier film 12.

The structures 16 include an inorganic layer 18 with a first major surface 17 and a second major surface 19. In various embodiments, the first major surface 17 of the inorganic layer 18 may be directly on the first major surface 13 of the flexible carrier film 12, or may be on an intermediate surface modifying layer as discussed in more detail below. In some example embodiments, the inorganic layer 18 has a thickness of less than about 200 nm, or less than about 100 nm, or less than about 50 nm.

The composition of the inorganic layer 18 may vary widely, but in some examples includes silicon oxides such as SiO₂, SiCₓO_{y} or SiAlₓO_{y}, as well as TiO, aluminum oxides AlOₓ, Au, and mixtures and combinations thereof. In contrast to traditional wafer processing, amorphous silicon oxide deposited by roll-to-roll processing may include impurities such as aluminum or carbon, which can make possible more efficient deposition rates on flexible, temperature sensitive surfaces using, for example, sputtering or PECVD technology.

In some embodiments, a silane with reactive functionality is condensed within the inorganic layer 18. The reactive functionality is selected to grow an analyte binding layer 20 on the inorganic layer 18, or to graft the analyte binding layer 20 to the inorganic layer 18. Suitable reactive functional groups for the silane include, but are not limited to, epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines and mixtures and combinations thereof. In some embodiments, the functional group is a photoreactive functional group such as benzophenone, aryl azide, halogenated aryl azide, diazos, or azos that can be used to grow or graft the analyte binding layer using radical chemistry. In some embodiments, norbornene silanes have been found to be particularly useful.

In some examples, the condensed reactive silane functionality is selected to provide a covalent bond at an interface between the second major surface 19 of the inorganic layer 18 and a first major surface 21 of the analyte binding layer 20. For example, the analyte binding layer 20 is covalently bound to the inorganic layer 18 through reaction with the condensed functional silane having any of the reactive functional groups listed above. Suitable examples of functional silanes include, but are not limited to, an acrylate silane, an aminosilane, an acrylamide silane, a norbornene silane, and mixtures and combinations thereof.

The reactive functional groups derived from the functional silane are separated from the inorganic layer 18 by hydrocarbon linking groups that more effectively bond the analyte binding layer 20 and the inorganic layer 18. The hydrocarbon linking group is at least one methylene unit long, and in various embodiments can include about 1 to about 20 carbon atoms, or about 2 to about 15 carbon atoms. In various embodiments, the hydrocarbon linking group can be linear, cyclic, branched, or aromatic, and can optionally include heteroatoms such as, for example oxygen, nitrogen, sulfur, phosphorus and combinations thereof.

The analyte binding layer 20 includes reactive functionality selected to bind with a target analyte. In some cases, the reactive functionality can be the same or different with respect to the reactive functionality used to covalently bind to the inorganic layer. In various embodiments, which are not intended to be limiting, the reactive functional groups within or on a second major surface 23 of the analyte binding layer are selected to bind biomolecules chosen from amino acids, nucleosides, nucleotides, peptides, oligonucleotides, polynucleotides, nucleic acids, proteins, carbohydrates, secondary metabolites, pharmaceutical molecules and mixtures and combinations thereof, as well as undesirable chemical contaminants found in liquid aqueous streams and water supplies. In some cases, the biomolecules are modified with chemistry that facilitate covalent attachment to the analyte binding layer. In some cases, the biomolecule can be used to bind additional analytes. For example, not intending to be limiting, the molecule is an oligonucleotide primer or a mixture of oligonucleotide primers that can bind complementary DNA or RNA molecules, an anti-body, or a carbohydrate that can bind a lectin.

In various embodiments, the analyte binding layer 20 is made of a functionalized material chosen from a reactive silane, a functionalizable hydrogel, a functionalizable polymer, and mixtures and combinations thereof. Suitable reactive functional groups for these functionalized materials include, but are not limited to, substituted and unsubstituted alkene, azide, alkyne, substituted and. substituted amine, carboxylic acid, substituted and unsubstituted hydrazone, halogen, hydroxy, substituted and unsubstituted tetrazole, substituted and unsubstituted tetrazine, thiol, epoxide, carbonyls included aldehyde and ketone, aziridines, oxiranes and combinations thereof. In one example, which is not intended to be limiting, DNA primer oligomers can be used which have alkynes that can be conjugated to an azide-functionalized hydrogel.

In some embodiments, the analyte binding layer 20 includes a polymer or hydrogel of Formula (Ia) or (Ib) below:

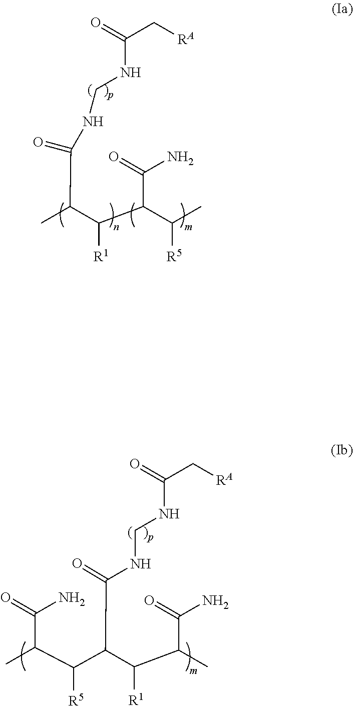

In Formulas 1a and 1b, R¹ is H or optionally substituted alkyl, the functional group R^{A} s selected from the group consisting of azide, optionally substituted amine, optionally substituted alkene, optionally substituted hydrazone, carboxylic acid, halogen, hydroxy, optionally substituted tetrazole, optionally substituted tetrazine, and thiol; R⁵ is selected from or optionally substituted all each of the —(CH₂)-p can be optionally substituted; p is an integer in the range of 1 to 50; n is an integer in the range of 1 to 50,000; and m is an integer in the range of 1 to 100,000. In some such embodiments, the functional groups include azides. In some embodiments, each R¹ and R⁵ is hydrogen. In some embodiments, the functional group R^{A} is azide. In some embodiments, p is 5. In one embodiment, the polymer or hydrogel included in the functionalizable layer is PAZAM. Methods for making and using PAZAM, and other functionizable materials that can be used in a layer of a substrate of the present disclosure are described in U.S. Pat. No. 9,012,022, the subject matter of which is incorporated herein by reference in its entirety.

Examples of reactive silanes that can be used include, but are not limited to, (meth)acrylate functional silanes, (meth)acrylamide functional silanes, aldehyde functional silanes, amino functional silanes, anhydride functional silanes, azide functional silanes, carboxylate functional silanes, phosphonate functional silanes, sulthnate functional slimes, epoxy functional silanes, ester functional silanes, vinyl functional silanes, olefin functional silanes, halogen functional silanes and dipodal silanes with any or none of the above functional groups. Norbomene silanes have been found to be particularly useful.

The choice of silane functionality can be made based on the reactivity of the material to which it will react. For example, the acrylamide or norbomene-functionalized silane can react with azide-functionalized polymers. Amino-functionalized silanes can reaction with carbon-functionalized polymer where the carbonyl is a carboxylic acid, an ester, an aldehyde, a ketone and activate ester and combinations thereof. Silanes with photoactive functionality such as benzophenones, diazos, or azidobenzyls can be used to graft any polymer with hydrocarbon linkages through hydrogen abstraction.

In some embodiments, the analyte binding layer 20 can include a hydrogel. Non-limiting examples of hydrogels are described in U.S. Pat. No. 9,012,022 and include polyacrylamide hydrogels and polyacrylamide hydrogel-based arrays. Other hydrogels are poly(meth)acrylate hydrogels and poly(methlacrylate-based arrays. Once hydrogels have been formed, biomolecules may then be attached to them to produce molecular arrays. The hydrogel may be modified chemically after it is produced. For example, the hydrogel may be polymerized with a co-monomer having a functionality primed or pre-activated to react with the biomolecules to he arrayed. In some examples, the array is formed at the same time as the hydrogel is produced by direct copolymerization of acrylafilide-derivatized polynucleotides. In one example, acrylamide phosphoramidite available from Mosaic Technologies, Boston, MA, under the trade designation ACRYDITE can be reacted with polynucleotides prior to copolymerization of the resultant monomer with acrylamide.

In some embodiments, the analyte binding layer 20 includes a polymer with one or more functional groups reactable with biomolecules of interest. In some such embodiments, the functional group can be chosen from substituted and unsubstituted alkene, azide, substituted or unsubstituted amine, carboxylic acid, substituted or unsubstituted hydrazone, halogen, hydroxy, substituted or unsubstituted tetrazole, substituted or unsubstituted substituted tetrazine, thiol, and combinations thereof.

In some embodiments, the polymer of Formula (Ia) or (Ib) is also represented by Formula (IIa) or (IIb):

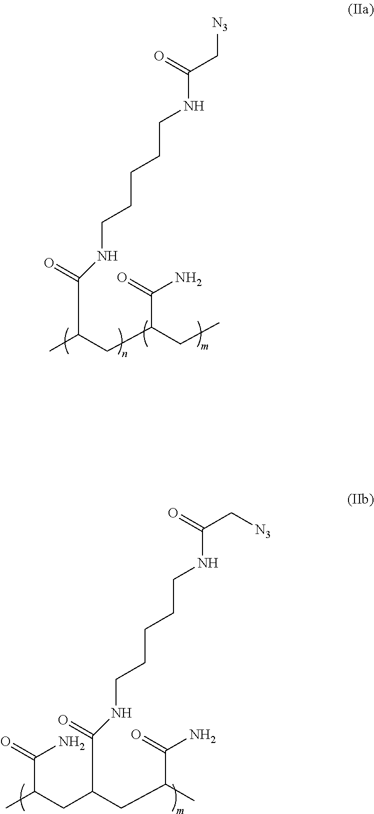

wherein n is an integer in the range of 1-20,000, and m is an integer in the range of 1-100,000.

In some embodiments, the functionalized molecule used for direct conjugation is poly(N-(5-azidoacetamidylpentyl)acrylamide-co-acrylamide) (PAZAM). PAZAM can be prepared by polymerization of acrylamide and Azapa (N-(5-(2-azidoacetamido)pentyl)acrylamide) in any ratio. In various embodiments, PAZAM is a linear polymer, a lightly cross-linked polymer, may be supplied in an aqueous solution, or may be supplied as an aqueous solution with one or more solvent additives. For example, in some embodiments the arylazide containing polymers described in US20190232890 may be used.

Referring again to FIG. 1, in some embodiments, the analyte binding layer 20 may include at least one photocurable polymer chosen from urethane, acrylate, silicone, epoxy, polyacrylic acid, polyacrylates, epoxysilicone, epoxy resins, polydimethysiloxane (PDMS), silsesquioxane, acyloxysilanes, maleate polyesters, vinyl ethers, monomers with vinyl or ethynyl groups, or copolymers and combinations thereof.

In some embodiments, the flexible carrier film 12 in the component article 10 includes an optional anti-biofouling layer 24 between the flexible carrier film 12 and the inorganic layer 18. For example, the anti-biofouling layer 24 can include a first major surface 25 contacting the flexible carrier film 12 and a second major surface 27 contacting the inorganic layer 18. In the embodiment of FIG. 1, which is not intended to be limiting, the structures 16 may include a base 26 formed from the anti-biofouling layer 24, and the inorganic layer 18 resides between the base 26 and the analyte binding layer 20. In such examples, the anti-biofouling layer 24 includes a surface 29 forming an interstitial region 28 between the structures 16.

The anti-biofouling layer 24 can include any material that resists or prevents accumulation or formation of biological species such as, for example, microorganisms, or biomolecules such as nucleic acids and proteins. The anti-biofouling layer 24 thus prevents target analytes, sequencing reagents or fluorophores from non-specifically adhering to at least a portion of the interstitial regions between the structures 16. If the anti-biofouling layer 24 is applied in a particular region of the component article 10, other regions uncoated by the anti-biofouling layer 24 may be bound with a biological sample. For example, if material from the anti-biofouling layer 24 is exposed on the top of the structures and absent from the regions between the structures, a biological sample can bind with an analyte binding material in the interstitial regions between the structures. In another example, if material from the anti-biofouling layer 24 is in the interstitial regions, and absent from exposed surfaces on top of the structures, the biological material can bind to an analyte binding material 20 on the tops of the structures. The anti-biofouling layer 24 thus provides specific placement of the analyte binding material (and the biological material bound thereto) in one or more areas of the component article 10.

In some examples, which are not intended to be limiting, suitable materials for the anti-biofouling layer 24 include fluorinated compounds such as fluoropolymers, non-aromatic hydrocarbon polymers, cyclic olefin polymers (COP), cyclic olefin copolymers, cyclic block copolymers, silicones with non-oxidized surface chemistry, and mixtures and combinations thereof. In some examples, the anti-biofouling layer includes an exposed upper layer of methyl groups deposited through plasma enhanced chemical vapor deposition (PECVD) of hexamethyldisiloxane.

In other embodiments, suitable materials for the anti-biofouling layer 24 can include metals, particularly noble metals such as Au, Ag, Pt, and alloys and mixtures thereof.

In some examples, which are not intended to be limiting, the anti-biofouling layer 24 is formed from a fluoropolymer available under the trade designation CYTOP from ACG Chemicals, Exton, MA, which are baked at high temperatures (greater than about 50° C.) for several 30 minute cycles. Other suitable materials for the antifouling layer 24 include fluorothermoplastics available under the trade designation THV from 3M Dyneon, St. Paul, MN, fluoropolymers such as HFPO, and those available under the trade designation LTM from Solvay, Alpharetta, GA.

In some examples, which are not intended to be limiting, the anti-biofouling layer includes a methyl terminated surface, which is rich in methyl groups. These surfaces are characterized by water contact angles greater than 100 degrees. In some examples, methyl groups can be formed from molecular fragmentation of hexamethyldisiloxane through plasma dissociation, although any method of creating a methyl-terminated surface may provide similar functionality. Other chemistries such as tetraethyl orthosilicate, tetramethylsilane, hexamethyldisilane, or trimethylamine may be deposited using plasma enhanced chemical vapor deposition to create methyl terminated surfaces. In addition, precursors such as trimethylamine may form a monolayer of methyl groups on an appropriate surface using atomic layer deposition.

In some embodiments, as shown schematically in FIG. 1, the second major surface 23 of the analyte binding layer 20 can be structured or roughened to more effectively bond with a target analyte (not shown in FIG. 1). For example, the roughness or structure of surface 23 can come from the surface roughness or structure of the layer 19 below it. In one embodiment, which is not intended to be limiting, the layer 19 can be modified by adding random nanostructures on the first major surface 17 by depositing a silicon containing discontinuous layer using plasma enhanced chemical vapor deposition (PECVD), while either simultaneously or sequentially etching the surface with a reactive species as described in U.S. Pat. Nos. 10,134,566 and 8,634,146, respectively, which are incorporated herein by reference in their entireties. The structured layer on the surface 19 of the inorganic layer 18 can optionally be overcoated with a thin layer of a silicon oxide to provide greater surface area for silane binding in the final construction. In various examples, the nanostructures can have characteristic length scales of about 5 nm to about 300 nm, and aspect ratio of 5:1 to 1:5 (height:width).

In some embodiments, the second major surface 15 of the flexible polymeric carrier layer 12 includes an optional adhesive layer 30. Any adhesive may be used in the adhesive layer 30, but low auto-fluorescent materials have been found to be particularly suitable for use in analytical devices for biochemical analytes. In some examples, which are not intended to be limiting, the adhesive layer 30 includes optically clear adhesives such as those available from 3M under the trade designation 3M OPTICALLY CLEAR ADHESIVE 8171, as well as polyisobutylene polymer adhesives. Suitable isobutylene adhesives can include styrene-isobutylene copolymers, or with multifunctional components such as (meth)acryl and vinyl ether groups.

In some examples, the adhesive layer 30 has a thickness of about 1 μm to about 50 μm, or about 5 μm to about 15 μm. In some embodiments, the adhesive layer 30 should be sufficiently uniform so that a focal plane of the exposed surfaces of the analyte binding layer 20 (second major surface 23 in FIG. 1) does not vary by more than about 5 μm, or more than about 2 μm, or more than about 1 μm, 500 nm, 250 nm or 100 nm.

A surface 33 of the adhesive layer 30 may optionally be structured with, for example, a network of air bleed channels, to reduce trapped air when the adhesive layer 30 is applied to a flat surface of a rigid substrate such as a glass plate. In some embodiments, the adhesive layer 30 may be a repositionable adhesive, and may optionally include glass beads, adhesives with low green strength, vacuum lamination, and the like.

The adhesive layer can be applied on the second major surface 15 of the flexible carrier film 12 using a wide variety of techniques including, coating directly on the surface 15, or via lamination of a transfer adhesive to the flexible substrate 12.

In some examples, the adhesive layer 30 is attached to an optional reinforcing layer or rigid substrate 32, which may provide increased rigidity so the component article 10 can be more readily used in commonly utilized in apparatus for performing biochemical assays. The reinforcing layer 32 may vary widely, and in various embodiments includes silicon, glass, plastic, metal, metal oxide, paper, and combinations thereof. In various embodiments, the reinforcing layer 32 may include a single layer or multiple layers. In some embodiments, a major surface 31 of the rigid substrate 30 may optionally be treated to enhance removal of the adhesive layer 30.

In another embodiment, the reinforcing layer 32 can be a release liner that protects the adhesive layer 30, and may be peeled away from the adhesive layer 30 such that the component article can be applied to a selected substrate prior to use in an apparatus for performing biochemical assays. Suitable release liners 32 include, but are not limited to, polymeric films, paper, metals, metal oxides, and combinations thereof. The release liner 32 may include single or multiple layers.

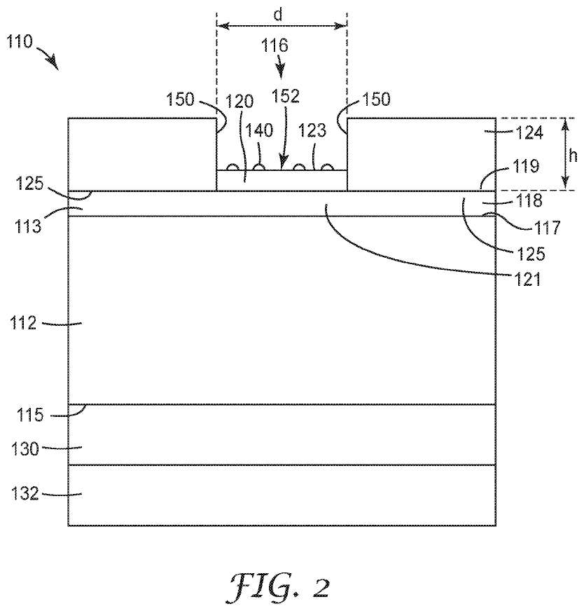

Referring now to FIG. 2, in another embodiment the layer materials and structures described with reference to FIG. 1 above may be arranged in a different manner to form a component article 110 including an arrangement of wells 116 including an analyte binding material, and a corresponding arrangement of structures 124, at least a portion of which are free of the analyte binding material. The device 110 of FIG. 2 includes a flexible carrier film substrate 112, which has a first major surface 113 and a second major surface 115.

An inorganic layer 118 includes a first major surface 117 and a second major surface 119, and the first major surface 117 resides on the first major surface 113 of the flexible carrier film 112. In some examples, as described above in the discussion of FIG. 1, the inorganic layer 118 can be include silicon oxides such as SiO₂, SiCₓO_{y} or SiAlₓO_{y}, TiO, AlOₓ, Au, and mixtures and combinations thereof.

In some embodiments, a silane with reactive functionality is condensed within the inorganic layer 118. The reactive functionality is selected to grow an analyte binding layer 120 on the inorganic layer 118, or to graft the analyte binding layer 120 to the inorganic layer 118. Suitable reactive functional groups for the silane include, but are not limited to, epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines and mixtures and combinations thereof. In some embodiments, the functional group is a photoreactive functional group such as benzophenone, aryl azide, halogenated aryl azide, diazos, or azos that can be used to grow or graft the analyte binding layer using radical chemistry. In some embodiments, norbornene silanes have been found to be particularly useful.

Optionally, in some embodiments a tie layer such as 3 aminopropyl triethoxysilane, poly(allyl) amine, aminopropylsilsesquioxanes available under the trade designation SILQUEST A-1106 from Momentive Performance Materials, Waterford, NY, bis-3-trimenthoxy silyl propylamine (available under the trade designation SILQUEST A-1170 from Momentive Performance Materials), diethylenetriaminopropylsilane (available under the trade designation SILQUEST A-1130 from Momentive Performance Materials), glycidoypropyltrimethoxysilane (available under the trade designation SILQUEST A-187 from Momentive Performance Materials, AP115, (a mixture of dilute (3-Glycidyloxypropyl)trimethoxysilane), or other amino silanes or amine-containing polymers, can be used to increase adhesion between the inorganic layer 118 and the analyte binding layer 120.

In another embodiment, the inorganic layer 118 can be structured by adding random nanostructures on the first major surface 113 of the flexible carrier film 112 by depositing a silicon containing discontinuous layer using plasma enhanced chemical vapor deposition while either simultaneously or sequentially etching the surface with a reactive species as described in U.S. Pat. Nos. 10,134,566 and 8,634,146, respectively. This layer can optionally be overcoated with a thin layer of silicon oxide to provide greater surface area for aminosilane binding in the final construction. The nanostrutured layer is advantageous for creating a mechanical binding mechanism.

The component article 110 further includes an anti-biofouling layer 124 residing on the second major surface 119 of the inorganic layer 118. The anti-biofouling layer 124 includes a first major surface 125 contacting the second major surface 119 of inorganic layer 118. As shown in the cross-sectional view of FIG. 2, portions of the anti-biofouling layer 124 extend beyond the analyte binding layer 120 to form the wells 116. The wells 116 thus include walls 150 and a floor 152 formed by the second major surface 123 of the analyte binding layer 120. In some cases (not shown in FIG. 2), the analyte binding layer 120 can bind to the walls 150 of the wells if the anti-biofouling walls are oxidized during etching and the reactive silane also binds there.

While the walls 150 of the wells 116 formed by the anti-biofouling layer 124 are shown as substantially perpendicular to the floor 152 to form wells 116 with a rectangular cross-sectional shape, in various embodiments the wells 116 can have a wide variety of cross-sectional shapes including, for example, trapezoidal, square, hemispherical, arcuate, and the like.

In various example embodiments, the walls have a height h above the second major surface 119 of the inorganic layer 118 of greater than 0 nm and up to 1000 nm, or between 50 nm and 200 nm. In various embodiments, the wells 116 have a diameter d of about 10 nm to about 10000 nm, or about 200 nm to about 700 nm.

As the analyte binding layer 120 is located at the floor 152, biochemical species bound to the analyte binding layer 120 reside within the wells 116, but do not bond with the structures 124. The wells 116 are separated by the walls 150 of the anti-biofouling layer 124, which confines the bound biochemical species within at least a portion of the individual wells 116 for further analysis.

In some examples, the floor 152 of the wells 116 (second major surface 123 of the analyte binding layer 120) may also include structures 140. As noted above in the discussion of FIG. 1, the structures 140 may extend away from the floors 152, or may form depressions in the floors 152. As discussed above with regard to FIG. 1, in some examples, structures in the surface 119 of the inorganic layer 118 can be carried over into the floors 152 of the wells 116.

In some embodiments, the second major surface 115 of the flexible polymeric carrier layer 112 includes an optional adhesive layer 130, which is some cases may be a low auto-fluorescent, optically clear material. In some embodiments, the adhesive layer 130 should be uniform so that a focal plane of the exposed surfaces of the wells 116 (second major surface 123 of the analyte binding layer 120 in FIG. 2) does not vary by more than about 5 μm, or more than about 2 μm, or more than about 1 μm, 500 nm, 250 nm or 100 nm.

In some examples, the adhesive layer 130 is attached to an optional reinforcing layer or rigid substrate 132, which may provide increased rigidity so the component article 110 can be more readily used in commonly utilized in apparatus for performing biochemical assays. In various embodiments, the reinforcing layer 132 can be a rigid material or a peelable release liner that exposes the adhesive layer 130 so that the component article 110 can be attached to a reinforcing layer for use in biochemical assay instrumentation or other analysis processes.

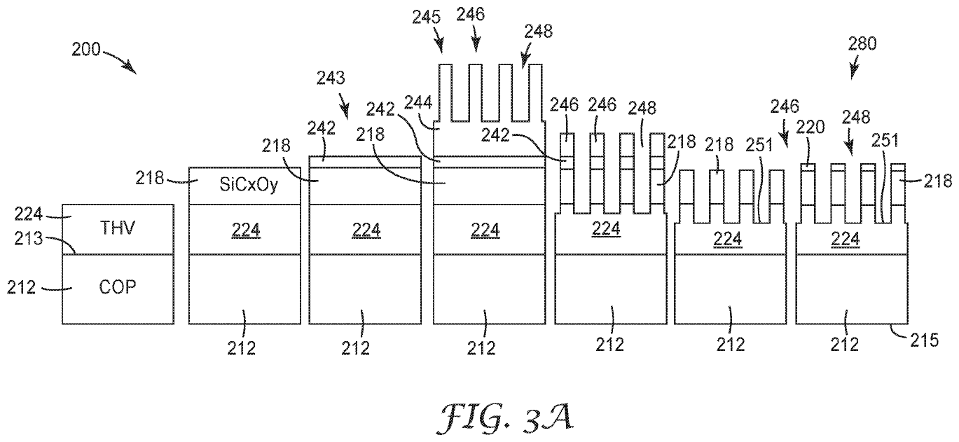

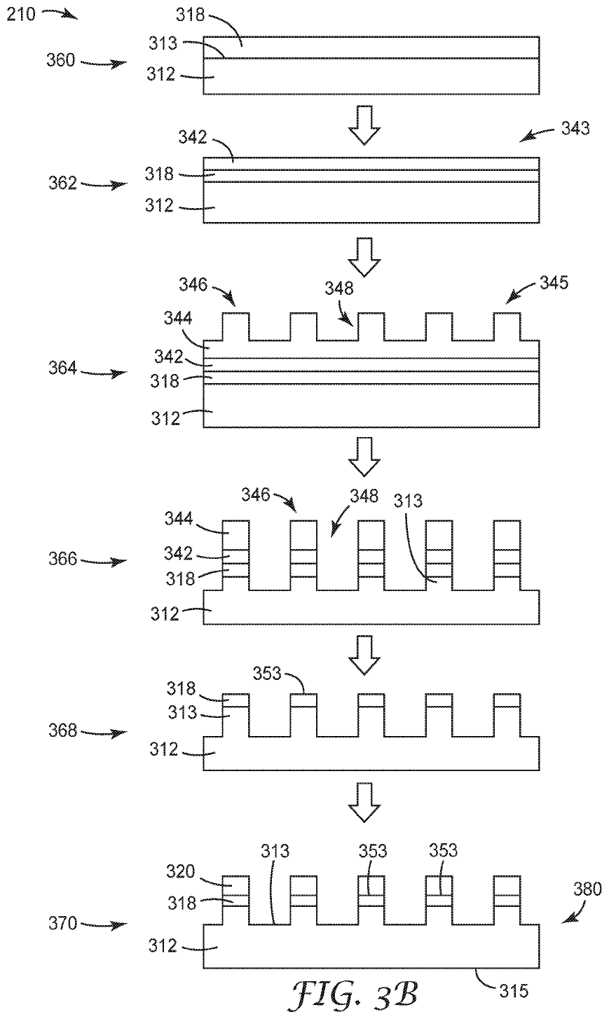

FIG. 3A is a schematic representation of an embodiment of process 200 for forming a component article with functionalized analyte binding posts exemplified by the article 10 of FIG. 1. In various embodiments, the process 200 may be performed on a production line in a roll-to-roll process to make a component article on a flexible carrier film substrate, or the individual articles may be individually produced.

In step 260, a flexible carrier film 212 is utilized as a low auto-fluorescent backing substrate for the component article. In this example, an optional anti-biofouling layer 224 is coated over at least a portion of a first major surface 213 of the flexible carrier film 212. For example, the anti-biofouling layer 224 may be solvent coated on the surface 213 and dried in a roll-to-roll process, or may be vapor coated on the surface 213. In various embodiments, the anti-biofouling layer 224 has a thickness of about 100 nm to about 1000 nm, or about 300 nm to about 500 nm.

In step 262 of the process 200, an inorganic layer 218, which also serves as an etch resist layer and in some examples has a composition of SiCₓO_{y} or SiAlₓO_{y}, is applied on the anti-biofouling layer 224. In some examples, the inorganic layer can be deposited on the anti-biofouling layer roll-to-roll by plasma enhanced chemical vapor deposition (PECVD) or sputtering. In various embodiments, the thickness of the inorganic layer 218 is about 5 nm to about 200 nm, or about 10 nm to about 50 nm.

In step 264, an optional silane tie layer 242 is applied on the inorganic layer 218 to form a construction 243. Suitable silane tie layers 242 include, for example, silane-modified polyvinyl alcohol blends such as 2-(3-trimethoxysilylpropylcarbamoyloxy)ethyl prop-2-enoate assembled as described in Example 7 of U.S. Pat. No. 9,790,396, which can be applied via a process such as solvent die coating.

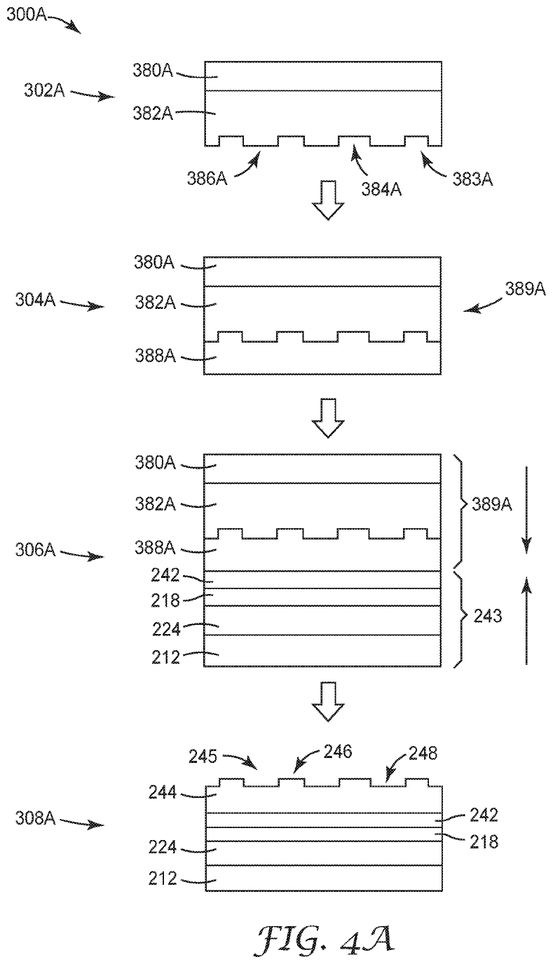

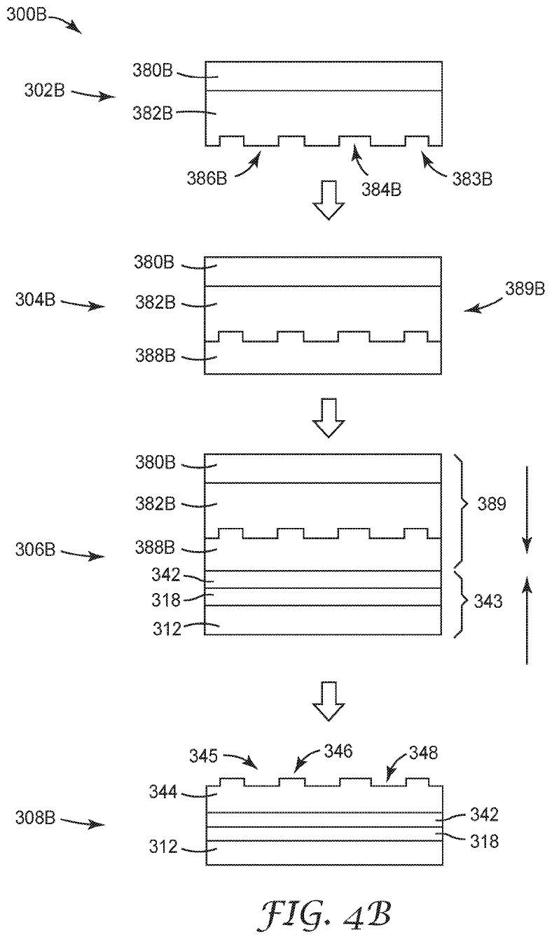

In step 266, a patterned masking layer 244 with a small residual layer is transferred onto the inorganic layer 218 as described in an embodiment of the process 300A of FIG. 4A. The steps of the process 300A may be conducted in a number of different sequences, and the order of steps in FIG. 4A is not intended to be limiting.

As shown in step 302A in FIG. 4A, an optional support layer 380A includes a patterned layer 382A. The patterned layer 382A includes a patterned surface 383A including one or more recessed features 384A, each recessed feature adjoining at least one plateau feature 386A.

In step 304A, a masking layer 388A is applied on the patterned surface 383A of the patterned layer 382A to form a transfer construction 389A.

In step 306A, the transfer construction 389A formed in step 304A is laminated to the construction 243 from step 264 of FIG. 3A. In the embodiment shown in step 306A, the masking layer 388A is contacted with the optional silane tie layer 242. However, in an alternative embedment (not shown in FIG. 3A), the masking layer 388A can be contacted with the inorganic layer 218. In another alternative embodiment (not shown in FIG. 3A), the masking layer 388A can initially be applied to either of the target substrates, the optional silane tie layer 242 or the inorganic layer 218. The transfer construction 389A can be free of the masking layer 388A, and the surface 383A of the patterned layer 382A can be contacted directly with the masking layer 388A in its position atop the target substrates 242 or 218.

In step 308A, the masking layer 388A of the transfer construction 389A is separated from the patterned layer 382A, leaving behind a patterned layer 244 with a patterned surface 245. The patterned surface 245 includes an arrangement of projections 246 interspersed with recessed features 248. The patterned surface 245 includes a pattern of projections 246 and recessed features 248 that is an inverse of the pattern of projections 386A and recessed features 384A in the surface 383A. The process 300A thus provides a low-land transfer of a patterned masking layer 244 to the construction 243, with the result shown in step 266 of FIG. 3A.

In one embodiment, the masking layer 244 is a UV-curable (meth)acrylate including a patterned surface 245 having an arrangement of posts 246 with diameters of about 100 nm to about 1500 nm, or about 200 nm and about 500 nm, which are interspersed with recessed features 248. In various embodiments, which are not intended to be limiting, the aspect ratio of the posts 246 in the patterned surface 245 is about 5:1 to about 1:5 (height:diameter), or about 2:1 and 1:1 (height:diameter). In some embodiments (not shown in FIG. 3A), the posts 246 can optionally be tapered with a taper angle of greater than 0° and less than about 25°, or about 2° to about 10°.

In step 268, the pattern from the patterned surface 245 is transferred to the anti-biofouling layer 224 using an etching step such as, for example, reactive ion etching using a fluorine compound. The depth of the etch can be controlled based on the duration and selectivity of the etch to remove the masking layer 244, the inorganic layer 218 and the anti-biofouling layer 224.

In step 270, an additional etch step with, for example, a fluorocarbon-rich etching material, is utilized to remove the remainder of the masking layer 244 and optional silane tie layer 242. The etching step also modifies the chemical composition of the exposed surface 251 of the anti-biofouling layer 224. For example, in one embodiment, an etching plasma can be used to form a thin amorphous mixing layer on the surface 251 that can vary in chemical composition as a function of the etching gas and process conditions. In some embodiments, oxygen-based etching chemistries will result in an oxidized, high surface energy, thin cross-linked layer on both silica and fluorinated substrates, while in other embodiments, fluorocarbon-rich etch chemistries can be used to maintain low surface energy top layers on the exposed surface 251, and high surface energies in exposed portions of the inorganic layer 218.

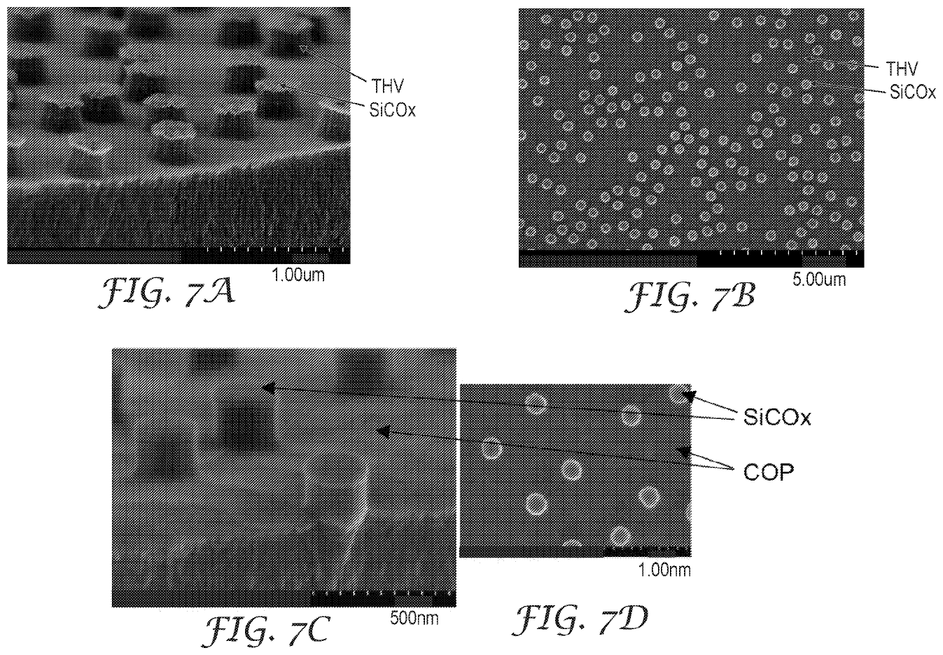

Photographs of an example construction of step 270 of FIG. 3A are shown in FIGS. 7A-7B.

In step 272, an analyte binding layer 220 of, for example, a functional alkoxy silane, overlies the inorganic layer 218 and bound thereto to form a component construction 280 for use in, for example, a biochemical assay. The analyte binding layer 220 does not react with the surface 251 within the recessed features 248 or other regions of the anti-fouling layer 224. In some examples, the analyte binding layer 220 may bond to portions of walls 250 of at least a portion of the posts 246.

In some example embodiments, the silane in the analyte binding layer 220 includes a reactive group that can be used to form a hydrogel polymer on the posts. In one example, which is not intended to be limiting, the alkoxysilane contains an acrylamide functional group. After post functionalization, the acrylamide in the analyte binding layer 220 is polymerized on the surface, leading to growth of poly(acrylamide) on the posts 246.

While not shown in FIG. 3A, as discussed above, in some embodiments, an optional adhesive layer may be applied on a second major surface 215 of the flexible carrier film 212 of the component construction 280. The adhesive layer may include an optional protective release liner, which may be removed so that the adhesive-backed component construction 280 may be affixed to a reinforcing layer such as glass, paper, a polymeric film, or the like.

FIG. 3B is a schematic representation of another embodiment of a process 210 for forming a component article with functionalized analyte binding posts exemplified by the article 10 of FIG. 1. In various embodiments, the process 210 may be performed on a production line in a roll-to-roll process to make a component article on a flexible carrier film substrate, or the individual articles may be individually produced.

In step 360, a flexible carrier film 312 is utilized as a low auto-fluorescent backing substrate for the component article. An inorganic layer 318, which also serves as an etch resist layer and in some examples has a composition of SiCₓO_{y} or SiAlₓO_{y}, is applied on a first major surface 313 of the flexible polymeric carrier file 312. In some examples, the inorganic layer can be deposited by roll-to-roll by plasma enhanced chemical vapor deposition (PECVD) or sputtering. In various embodiments, the thickness of the inorganic layer 218 is about 5 nm to about 200 nm, or about 10 nm to about 50 nm. The inorganic layer 218 may be continuous or discontinuous.

In step 362, an optional silane tie layer 342 is applied on the inorganic layer 318 to form a construction 343. Suitable silane tie layers 342 include, for example, silane-modified polyvinyl alcohol blends such as 2-(3-trimethoxysilylpropylcarbamoyloxy)ethyl prop-2-enoate assembled as described in Example 7 of U.S. Pat. No 9,790,396, which can be applied via a process such as solvent die coating. In another embodiment, the inorganic layer 318 may be roughened as described above in the discussion of FIG. 1.

In step 364, a patterned masking layer 344 with a small residual layer is transferred onto the inorganic layer 318 as described in the process 300B of FIG. 4B. The steps of the process 300B may be conducted in a number of different sequences, and the order of steps in FIG. 4B is not intended to be limiting.

As shown in FIG. 4B, an optional support layer 380B includes a patterned layer 382B. The patterned layer 382B includes a patterned surface 383B including one or more recessed features 384B, each recessed feature adjoining at least one plateau feature 386B.

In step 304B, a masking layer 388B is applied on the patterned surface 383B of the patterned layer 382B to form a transfer construction 389B.

In step 306B, the transfer construction 389B formed in step 304B is laminated to the construction 343 from step 362 of FIG. 3B. In step 306B, the transfer construction 389B formed in step 304B is laminated to the construction 343 from step 364 of FIG. 3B. In the embodiment shown in step 306B, the masking layer 388B is contacted with the optional silane tie layer 342. However, in an alternative embedment (not shown in FIG. 3B), the masking layer 388B can be contacted with the inorganic layer 318. In another alternative embodiment (not shown in FIG. 3B), the masking layer 388B can initially be applied to either of the target substrates, the optional silane tie layer 342 or the inorganic layer 318. The transfer construction 389B can be free of the masking layer 388B, and the surface 383B of the patterned layer 382B can be contacted directly with the masking layer 388B in its position atop the target substrates 342 or 318.

In step 308B, the masking layer 388B of the transfer construction 389B is separated from the patterned layer 382B, leaving behind a patterned layer 344 with a patterned surface 345. The patterned surface 345 includes an arrangement of projections 346 interspersed with recessed features 348. The patterned surface 345 includes a pattern of projections 346 and recessed features 348 that is an inverse of the pattern of projections 386B and recessed features 384B in the surface 383B. The process 300B thus provides a low-land transfer of a patterned masking layer 344 to the construction 343, with the result shown in step 366 of FIG. 3B.

In one embodiment, the patterned masking layer 344 is a UV-curable (meth)acrylate including a patterned surface 345 having an arrangement of posts 346 with diameters of about 100 nm to about 1500 nm, or about 200 nm and about 500 nm, which are interspersed with recessed features 348. In various embodiments, which are not intended to be limiting, the aspect ratio of the posts 346 in the patterned surface 345 is about 10:1 to about 1:70 (height:diameter), or about 5:1 to about 1:5, or about 2:1 and 1:1. In some embodiments (not shown in FIG. 3B), the posts 346 can optionally be tapered with a taper angle of greater than 0° and less than about 25°, or about 2° to about 10°.

In step 366, an etch is utilized to remove portions of the patterned masking layer 344, the silane tie layer 342, and the inorganic layer 318 from between the projections 346. The etching step forms an arrangement of posts 346 extending upward from the surface 313 of the flexible carrier film 312. For example, a reactive ion etching using a fluorine compound can be applied to remove the portions of the inorganic layer 318 and expose the surface 313 of the flexible carrier film 312. The depth of the etch can be controlled based on the selectivity an duration of the etch to remove the masking layer 344, the silane tie layer 342, the inorganic layer 318, and the flexible carrier film 312.

In step 368, an additional etch step is utilized to remove the remainder of the patterned masking layer 344 and the silane tie layer 342 and expose surfaces 353 at the tops of the posts 346. In some embodiments, oxygen-based etching chemistries will result in an oxidized, high surface energy, thin cross-linked layer on the surfaces 353. In another embodiment, a fluorocarbon-rich etch can be used to enhance or maintain the anti-biofouling properties of exposed surfaces of the recessed features 348.

Photographs of a construction of step 368 made according to the process of FIG. 3B are shown in FIGS. 7C-7D.

In step 370, an analyte binding layer 320 of, for example, a functional alkoxy silane, is coated on the inorganic layer 318 and bound thereto to form a component construction 380 for use in, for example, a biochemical assay. This analyte binding layer 320 does not react with the surfaces 313 interspersed with the posts 346, but preferentially bonds to the surfaces 353 on the tops of the posts 346. In some example embodiments, the silane in the analyte binding layer 320 include a reactive group that can be used to form a hydrogel polymer on the posts. In one example, which is not intended to be limiting, the alkoxysilane contains an acrylamide functional group. After post functionalization, the acrylamide in the analyte binding layer 320 is polymerized on the surface, leading to growth of poly(acrylamide) on the posts 346.

While not shown in FIG. 3B, as discussed above, in some embodiments, an optional adhesive layer may be applied on a second major surface 315 of the flexible carrier film 312 of the component construction 380. The adhesive layer may include an optional protective release liner, which may be removed so that the adhesive-backed component construction 380 may be affixed to a reinforcing layer such as glass, paper, a polymeric film, or the like.

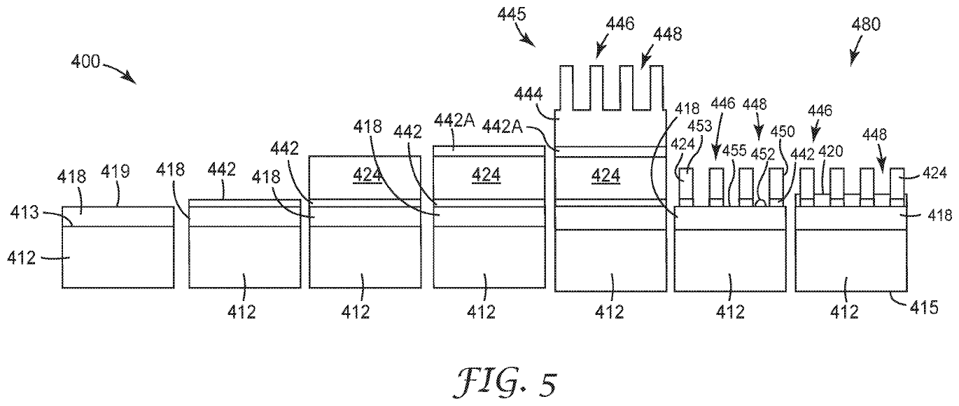

Referring now to FIG. 5, a schematic representation of an embodiment of process 400 is shown for forming a component article with functionalized analyte binding wells exemplified by the article 110 of FIG. 2. In various embodiments, the process 400 may be performed on a production line in a roll-to-roll process to make a component article on a flexible carrier film substrate, or the individual articles may be individually produced.

In step 460, a flexible carrier film 412 is utilized as a low auto-fluorescent backing substrate for the component article. An inorganic layer 418, which also serves as an etch resist layer and in some examples has a composition of SiCₓO_{y} or SiAlₓO_{y}, is applied on at least a portion of a first major surface 413 of the flexible carrier film 412. In some examples, the inorganic layer 418 can be deposited on the flexible polymeric film substrate 412 by plasma enhanced chemical vapor deposition (PECVD) or sputtering. In various embodiments, the thickness of the inorganic layer 418 is about 5 nm to about 200 nm, or about 10 nm to about 50 nm.

In step 462, an optional first tie layer 442 is applied on the inorganic layer 418. Suitable materials for the first tie layer 442 include, but are not limited to, 3-aminopropyl triethoxysilane, poly(allyl) amine, aminopropylsilsesquioxane, bis-3-trimenthoxy silyl propylamine, diethylenetriaminopropylsilane, or glycidoypropyltrimethoxysilane, or those available under the trade designation AP115 (a mixture of dilute (3-glycidyloxypropyl)trimethoxysilane), or other amino silanes or amine-containing polymers. The first tie layer 442 can be used to increase adhesion between the subsequently applied anti-biofouling layer and the inorganic layer 418.

In some example embodiments, the application of the first tie layer 442 can be eliminated by structuring an exposed surface 419 of the inorganic layer 418. The exposed surface 419 can be modified by adding random nanostructures on the first major surface 413 if the flexible carrier film 412 by depositing a silicon containing discontinuous layer using plasma enhanced chemical vapor deposition (PECVD) while either simultaneously or sequentially etching the surface with a reactive species as described in U.S. Pat. Nos. 10,134,566 and 8,634,146, respectively, which are incorporated herein by reference in their entireties. The structured layer on the surface 419 of the inorganic layer 418 can optionally be overcoated with a thin layer of a silicon oxide (not shown in FIG. 5) to provide greater surface area for aminosilane binding in the final construction. This nanostrutured layer is advantageous for creating a mechanical binding mechanism between the inorganic layer 418 and the subsequently applied anti-biofouling layer 424 (shown in step 464 of FIG. 5).

In another embodiment, the exposed surface 419 of the inorganic layer 418 can be structured by molding a layer of a UV-curable or thermoplastic material while the layer is in contact with the first major surface 413 of the flexible carrier film 412. The inorganic layer 418 is then applied over the molded layer, which then creates a similar pattern of structures in the inorganic layer 418.

In step 464, an anti-biofouling layer 424 is applied on the first silane tie layer 442. For example, the anti-biofouling layer 424 may be solvent coated and dried in a roll-to-roll process, or may be vapor coated. In various embodiments, the anti-biofouling layer 424 has a thickness of about 100 nm to about 1000 nm, or about 300 nm to about 500 nm.

In step 466, a second tie layer 442A can optionally be applied to the anti-biofouling layer 424 to increase adhesion of the anti-biofouling layer 424 to subsequently applied layers. For example, if the anti-biofouling layer 424 includes a fluoropolymer, in one embodiment a very thin layer (having a thickness of about 10 nm to about 300 nm) of a poly(allyl) amine can be applied to the anti-biofouling layer 424 to enhance adhesion to subsequently applied acrylates. In another embodiment, a washable second tie layer 442A such as PVA (poly(vinyl alcohol)) with a thickness of about 10 nm to about 300 nm can be used.

In step 468, a patterned masking layer 444 is transferred onto the anti-biofouling layer 424 as described in FIGS. 4A-4B. In one embodiment, the patterned masking layer 444 is a UV-curable (meth)acrylate including a patterned surface 445 having an arrangement of plateau features 446 forming therebetween recessed wells 448 with diameters of about 100 nm to about 1500 nm, or about 200 nm and about 500 nm. In various embodiments, which are not intended to be limiting, the aspect ratio of the pattern the patterned surface 445 is about 10:1 to about 1:70 (height:diameter), or about 5:1 to about 1:5, or about 2:1 and 1:1. In some embodiments (not shown in FIG. 5), the plateau features 446 can optionally be tapered with a taper angle of greater than 0° and less than about 25°, or about 2° to about 10°.

In step 470, the pattern from the patterned surface 445 is transferred to the anti-biofouling layer 424 using an etching step such as, for example, reactive ion etching. The depth of the etch down to the inorganic layer 418 can be controlled based on the relative thicknesses of the masking layer 444, the first tie layer 442, and portions of the anti-biofouling layer 424. The etch exposes the inorganic layer 418 at the bottoms of the wells 448, which are separated by plateau-like land areas 447 derived from the anti-biofouling layer 424. The wells 448 include walls 450 formed by the land areas 447 that in some embodiments are substantially normal to bottoms or floors 455 of the wells 448 derived from the inorganic layer 418. In some embodiments, the walls 450 may be tapered with a taper angle of greater than 0° and less than about 25°, or about 2° to about 10°. The etch chemistry and conditions are chosen to avoid oxidizing the exposed portions of the anti-biofouling layer 424, yielding a thin amorphous layer of fluorocarbon in the substantially planar surfaces 453 on the land areas 447, while maintaining the high surface energy of the exposed surfaces 455 of the inorganic layer 418 residing in the bottom of the wells 448.

In some embodiments, as described above, all or a portion of the exposed surfaces 455 can optionally include structures 452 to enhance adhesion to subsequently applied layers.

In step 472, an analyte binding layer 420 of, for example, a functional alkoxy silane, is coated on the surfaces 455 of the inorganic layer 418 on the bottoms or floors of the wells 448 and bound thereto to form a component construction 480 for use in, for example, a biochemical assay. This analyte binding layer 420 does not react with the surfaces 453 on the land areas 447. In some example embodiments, the silane in the analyte binding layer 420 includes a reactive group that can be used to form a hydrogel polymer on the surfaces 455 at the bottoms of the wells 448. In one example, which is not intended to be limiting, the alkoxysilane contains an acrylamide functional group. After functionalization, the acrylamide in the analyte binding layer 420 is polymerized on the surface 449, leading to growth of poly(acrylamide) on the floors 455 of the wells 448.

While not shown in FIG. 5, as discussed above, in some embodiments, an optional adhesive layer may be applied on a second major surface 415 of the flexible carrier film 412 of the component construction 480. The adhesive layer may include an optional protective release liner, which may be removed so that the adhesive-backed component construction 480 may be affixed to a reinforcing layer such as glass, paper, a polymeric film, or the like.

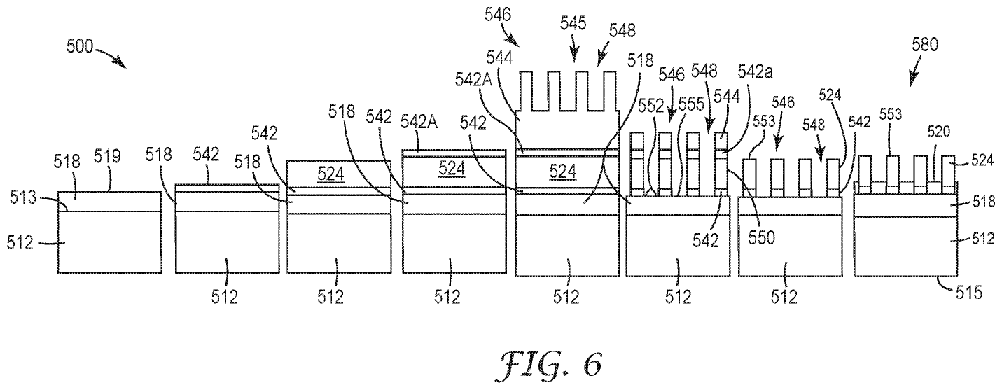

FIG. 6 schematically represents another embodiment of a process 500 for forming a

component article with functionalized analyte binding wells exemplified by the article 110 of FIG. 2. In various embodiments, the process 500 may be performed on a production line in a roll-to-roll process to make a component article on a flexible carrier film substrate, or the individual articles may be individually produced.

In step 560, a flexible carrier film 512 is utilized as a low auto-fluorescent backing substrate for the component article. An inorganic layer 518, which also serves as an etch resist layer and in some examples has a composition of SiCₓO_{y} or SiAlₓO_{y}, is applied on at least a portion of a first major surface 513 of the flexible carrier film 512. In some examples, the inorganic layer 518 can be deposited on the flexible polymeric film substrate 512 by plasma enhanced chemical vapor deposition (PECVD) or sputtering. In various embodiments, the thickness of the inorganic layer 518 is about 5 nm to about 200 nm, or about 10 nm to about 50 nm.

In step 562, an optional first tie layer 542 is applied on the surface 519 of the inorganic layer 518. Suitable materials for the first tie layer 542 include, but are not limited to, 3-aminopropyl triethoxysilane, poly(allyl) amine, aminopropylsilsesquioxane, bis-3-trimenthoxy silyl propylamine, diethylenetriaminopropylsilane, or glycidoypropyltrimethoxysilane, or those available under the trade designation AP115 (a mixture of dilute (3-glycidyloxypropyl)trimethoxysilane), or other amino silanes or amine-containing polymers. The first tie layer 542 can be used to increase adhesion between the subsequently applied anti-biofouling layer and the inorganic layer 518.

In some example embodiments, the application of the first tie layer 542 can be eliminated by structuring an exposed surface 519 of the inorganic layer 518. The exposed surface 519 can be modified by adding random nanostructures on the first major surface 513 if the flexible carrier film 512 by depositing a silicon containing discontinuous layer using plasma enhanced chemical vapor deposition (PECVD) while either simultaneously or sequentially etching the surface with a reactive species as described in U.S. Pat. Nos. 10,134,566 and 8,634,146, respectively, which are incorporated herein by reference in their entireties. The structured layer on the surface 519 of the inorganic layer 518 can optionally be overcoated with a thin layer of a silicon oxide (not shown in FIG. 6) to provide greater surface area for aminosilane binding in the final construction. This nanostrutured layer is advantageous for creating a mechanical binding mechanism between the inorganic layer 518 and the subsequently applied anti-biofouling layer 524 (shown in step 564 of FIG. 6).

In another embodiment, the exposed surface 519 of the inorganic layer 518 can be structured by molding a layer of a UV-curable or thermoplastic material while the layer is in contact with the first major surface 513 of the flexible carrier film 512. The inorganic layer 518 is then applied over the molded layer, which then creates a similar pattern of structures in the inorganic layer 518.

In step 564, an anti-biofouling layer 524 is applied on the first silane tie layer 542. For example, the anti-biofouling layer 524 may be solvent coated and dried in a roll-to-roll process, or may be vapor coated. In various embodiments, the anti-biofouling layer 524 has a thickness of about 10 nm to about 300 nm, or about 10 nm to about 150 nm.

In step 566, a second tie layer 542A is applied to the anti-biofouling layer 524 to increase adhesion of the anti-biofouling layer 524 to subsequently applied layers and to form a subsequent peelable layer as explained in more detail below. For example, if the anti-biofouling layer 524 includes a fluoropolymer, in one embodiment a very thin layer (having a thickness of about 10 nm to about 300 nm) of a poly(allyl) amine can be applied to the anti-biofouling layer 524 to enhance adhesion to subsequently applied acrylates. In another embodiment, a washable second tie layer 542A such as PVA (poly(vinyl alcohol)) with a thickness of about 10 nm to about 300 nm can be used.

In step 568, a patterned masking layer 544 is transferred onto the anti-biofouling layer 524

as described in the processes 300A-300B of FIGS. 4A-4B. In one embodiment, the patterned masking layer 544 is a UV-curable (meth)acrylate including a patterned surface 545 having an arrangement of plateau features 546 forming therebetween wells 548 having diameters of about 100 nm to about 1500 nm, or about 200 nm and about 500 nm. In various embodiments, which are not intended to be limiting, the aspect ratio of the pattern in the patterned surface 545 is about 10:1 to about 1:70 (height:diameter), or about 5:1 to about 1:5, or about 2:1 and 1:1. In some embodiments (not shown in FIG. 6), the plateau features 546 can optionally be tapered with a taper angle of greater than 0° and less than about 25°, or about 2° to about 10°.

In step 570, the pattern from the patterned surface 545 is transferred to the anti-biofouling layer 524 using an etching step such as, for example, reactive ion etching. The depth of the etch down to the inorganic layer 518 can be controlled based on the relative thicknesses of the masking layer 544, the first tie layer 542, portions of the anti-biofouling layer 524, and the second tie layer 542A. The etch exposes the inorganic layer 518 at the bottoms of the wells 548, which are separated by the plateau features 546 derived from the anti-biofouling layer 524 and the second tie layer 542A, and optionally the masking layer 544. The wells 548 include walls 550 formed by the surrounding plateau features 546 that are substantially normal to the bottoms or floors 555 derived from the inorganic layer 518. In some embodiments, the walls 550 may be tapered with a taper angle of greater than 0° and less than about 25°, or about 2° to about 10°. The etch chemistry and conditions are chosen to oxide the exposed portions of the inorganic layer 518, to maintain the high surface energy of the exposed surfaces 555 of the inorganic layer 518 residing in the bottom of the wells 548.

In some embodiments, the exposed top surfaces of the patterned masking layer 445 or the second tie layer 542A can optionally be nanostructured as described in U.S. Pat. Nos. 8,634,146, 10,134,566, and 9,908,772, each incorporated by reference herein in their entireties, to increase the adhesion in a subsequent peel step.

In some embodiments, as described above, all or a portion of the exposed surfaces 555 can of the inorganic layer 518 can optionally include structures 552 to enhance adhesion to subsequently applied layers.

In step 572, the second tie layer 542A and any remaining masking layer 544 are removed by using either an adhesive or coating an acrylate (optionally in solvent), curing while in contact with a carrier film, then peeling onto the carrier film. Heat and corona can optionally be used to increase adhesion of the tops 553 of the plateau features 546 and the adhesive or acrylate. In some embodiments, water can be used to remove acrylates described in WO2018/005311 A1 or WO2016/176129 A1. In some embodiments, hot water and/or ultrasonics can be used to remove poly(allyl)amine or PVA.

In step 574, an analyte binding layer 520 of, for example, a functional alkoxy silane, is coated on the surfaces 555 of the inorganic layer 518 on the bottoms or floors of the wells 548 and bound thereto to form a component construction 580 for use in, for example, a biochemical assay. The analyte binding layer 520 does not react with the surfaces 553 at the tops of the plateau features 546. In some example embodiments, the silane in the analyte binding layer 520 includes a reactive group that can be used to form a hydrogel polymer on the surfaces 555 at the bottoms of the wells 548. In one example, which is not intended to be limiting, the alkoxysilane contains an acrylamide functional group. After functionalization, the acrylamide in the analyte binding layer 520 is polymerized on the surface, leading to growth of poly(acrylamide) on the floors 555 of the wells 548.

While not shown in FIG. 6, as discussed above, in some embodiments, an optional adhesive layer may be applied on a second major surface 515 of the flexible carrier film 512 of the component construction 580. The adhesive layer may include an optional protective release liner, which may be removed so that the adhesive-backed component construction 580 may be affixed to a reinforcing layer such as glass, paper, a polymeric film, or the like.

The components and devices described above can be used in a wide variety of biochemical analysis procedures including, but not limited to, DNA sequencing tests. For example, a diagnostic device for DNA sequencing can include a flow cell with a patterned arrangement of fluidic channels configured to provide flow conduits for a sample fluid with a target analyte including polynucleotides and nucleic acids. At least some of the fluidic channels of the flow cell can be lined on a surface thereof with posts or wells including an analyte binding layer bonded to an underlying Si oxide layer by a network of methylene groups. The target analyte in the sample fluid is bound on the analyte binding layer, and the bound target analyte is exposed to a fluorescent reagent such that the analyte is detectable using spectroscopy.

In another example, the diagnostic device could be included in a DNA sequencing kit, a kit for detection of an environmental contaminant, a kit for detection of a particular viral or bacterial pathogen, and the like. The kit can include the diagnostic device along with reagents selected for the particular assay to be performed with the diagnostic device, such as fluorescent reagents, as well as appropriate instructions for use of the diagnostic device to conduct a particular assay or group of assays.

The devices of the present disclosure will now be further described in the following non-limiting examples.

### EXAMPLES

Unless otherwise noted or readily apparent from the context, all parts, percentages, ratios, etc. in the Examples and the rest of the specification are by weight.

### Materials

Materials and reagents utilized in the examples below are set forth in Table 1.

TABLE 1
Materials

| Abbreviation | Description and Source |
|---|---|
| Fluoropolymer | 5 wt % THV220G, in a 80/20 solution of MEK/MIBK |
| Solution 1 | |
| Acrylate | 13 wt % Acrylate resin A, 1 wt % HFPO-UA solution, |
| Solution A | 43 wt % MEK, 43 wt % PGME |
| Acrylate Resin A | 75 wt % Photomer 6210 with 25 wt % SR238 and 0.5% TPO |
| PHOTOMER 6210 | Urethane acrylate oligomer PHOTOMER 6210; IGM Resins, |
| | Charlotte, NC |
| SR238 | 1,6-Hexandiol diacrylate; SR238 Sartomer Americas, Exton, PA |
| SR351 | Trimethylopropane triacrylate; SR351, Sartomer Americas |
| IPA | Isopropyl alcohol; Brenntag Great Lakes, Wauwatosa, WI |
| MEK | Methyl ethyl ketone from Brenntag Great Lakes |
| PGME | Propylene Glycol Methyl Ether; Brenntag Great Lakes |
| MIBK | Methyl isobutyl ketone; Brenntag Great Lakes |
| THV 220G | THV; Dyneon THV 220G; 3M Co. Saint Paul, MN |
| TPO | Diphenyl(2,4,6-trimethylbenzoyl)phosphine |
| | Oxide; IRGACURE TPO; BASF, Florham Park, NJ |
| HFPO-UA Solution | 66% Hexafluoropropyleneoxide multiacrylate in Acetone; 3M Co. |
| Polyallyl amine | 15% aqueous solution of 15,000 MW Polyallyl amine; |
| | Polysciences Inc, Warrington PA |
| Bis-3-trimenthoxy | Silquest A-1170, Momentive, Waterford, NY |
| silyl propylamine | |
| K90 | 2-(3-trimethoxysilylpropylcarbamoyloxy)ethyl prop-2-enoate; 3M Co., |
| | assembled as described in Example 7 of U.S. Pat. No. 9,790,396 |
| Polycarbonate Film | 125-micron thick polycarbonate film with a gloss surface finish on both |
| | sides; Tekra, Inc. New Berlin, WI |
| Melenex 454 | Polyester film; MELENEX 454; Tekra, Inc. |
| ST505 | Polyester film; MELENEX ST505; Tekra, Inc. |
| COP Film | Zeonor Film, Zeon Chemicals LP, San Jose, CA |
| 3M TM Optically | 25 micron optically clear laminating adhesive |
| Clear Adhesive | |
| 8171 | |
| Adhesive Group A | Examples disclosed in US20200095476 |
| Adhesive Group B | Examples disclosed in US20200048511 |
| Adhesive Group C | Examples disclosed in US patent application No. 62/851,326 |
| Tie Layer 1 | 0.3% Bis-3-trimenthoxy silyl propylamine (Silquest A-1170) in MEK |
| Tie layer 2 | 0.45% poly(allyl) amine in 2.55% water and 97% IPA) |
| Tie layer 3 | 0.3% K90 in MEK |
| O₂ | Oxygen (UHP compressed gas) |
| | Oxygen Service Company, Saint Paul, MN |
| HMDSO | Hexamethyldisiloxane; Gilest Inc., Morrisville, PA |
| C₆F₁₄ | 3M PF-5060; 3M Co. |
| 3M Optically Clear | 125 micron optically clear, double-sided adhesive |
| Adhesive 8187 | |
| 1M Tris-HCl, pH | Invitrogen UltraPure 1M Tris-HCI Buffer, pH 7.5, Thermo Fisher |
| 7.5 | Scientific, Waltham, MA |
| HTI Hybridization | HTI hybridization buffer, Illumina 15027041 |
| Buffer | |
| SYBR Gold Dye | Invitrogen SYBR Gold Nucleic Acid Gel Stain (10,000X Concentrate in |
| | DMSO), Thermo Fisher Scientific, S11494 |
| 96-well Clear | Nunc MicroWell 96-Well Optical-Bottom Plate with Polymer Base and |
| Bottom Plate | solid polystyrene black upper structures with a polystyrene film at the |
| | bottom of the plate, Thermo Fisher Scientific, 165305 |
| Sealing Film for | Applied Biosystems MicroAmp Optical Adhesive Film Kit, Thermo |
| 96-well Plates | Fisher Scientific, 4313663 |
| Quinine | Alfa Aesar, Ward Hill, MA |
| hemisulfate | |
| monohydrate | |
| Resolve ™ | Microscope Immersion Oil from Criterion Sciences under Cornwell |
| | Corp., Riverdale, NJ |
| APTMS | 3-aminopropyltrimethoxysilane from Alfa Aesar, Ward Hill, MA |
| EtOH | Anhydrous ethanol from EMD Millipore, Burlington, MA |
| HOAc | Acetic acid from EMD Millipore, Billerica, MA |
| AF488 NHS Ester | Alexa FluorTM 488 NHS ester (succinimidyl ester) from Thermo Fisher |
| | Scientific, Waltham, MA |
| TE Buffer pH 8 | Invitrogen ™ TE Buffer pH 8 from Thermo Fisher Scientific, Vilnius, |
| | Lithuania |

### TEST METHODS

### Test Method 1: (DNA Adherence)

Step 1: A Portion of sample under test was cut, and one side of a double-sided adhesive was applied to the untreated side of the sample.
Step 2: Punches of samples were obtained (5 mm) and the release liner from back of adhesive was removed with fine tip forceps
Step 3: Punches from each sample were attached to the bottom of a 96-well clear bottom plate with the treated side of the same facing up.
Step 4: A DNA mixture was prepared to treat the samples. Five hundred microliters of undiluted, pooled sequencing library (average 15 nM concentration per sample) that was generated using the Illumina Nextera DNA flex library preparation kit (Illumina 20018704) was denatured with 50 μl of 1N NaOH for 5 min.

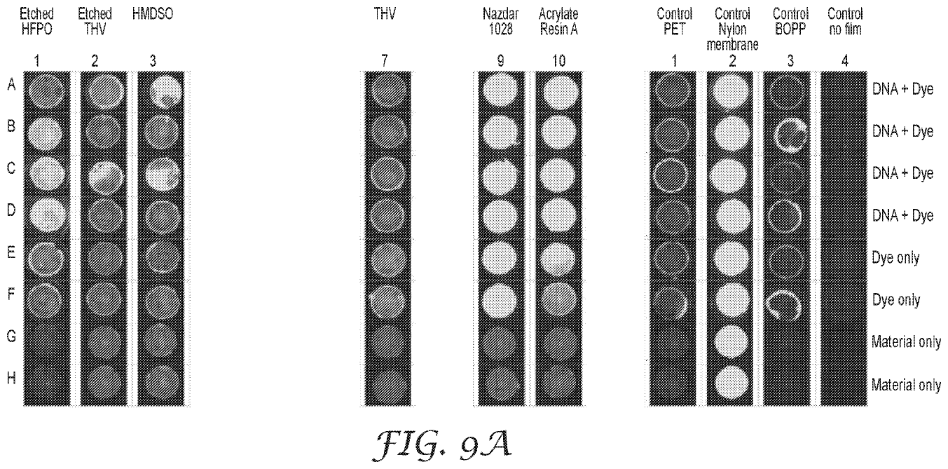

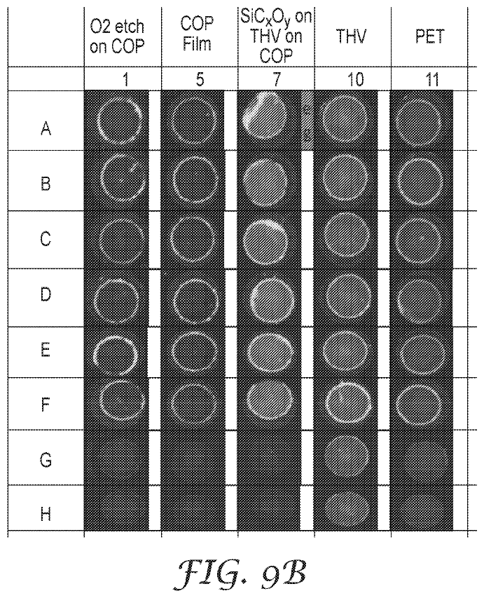

Fifty microliters of 1M Tris-HCl pH 7.5 was added to the DNA to neutralize, and mixture was vortexed briefly. DNA library was further denatured by heating at 95° C. for 3 min, then snap-cooled on ice. The denatured pooled DNA library was added to 4.5 ml of HT1 hybridization buffer, and the mixture was vortexed at high speed for 30 sec.
Step 5: The mixture from Step 4 was transferred to a reagent reservoir and a multichannel pipet was used to transfer 75 μl into each sample well in the 96-well plate. 75 μl allowed for flooding of entire sample with liquid. Material+dye controls or material only controls received 75 μl of HTI hybridization buffer without DNA.
Step 6: The 96-well plate was securely covered with sealing film and incubated at RT for 1 hour
Step 7: Liquid was Removed using a Multichannel Pipet
Step 8: One hundred microliters of 20 mM Tris-HCl pH 7.5 containing 1×SYBR gold dye was added to the wells n=4 (rows A-D in FIGS. 9A-9B) and material+dye controls n=2 per sample (rows E and F in FIGS. 9A-9B).
Step 9: One hundred microliters of 20 mM Tris-HCl pH 7.5 without dye added to material only wells n=2 per sample.
Step 10: Fluorescence readings were obtained using an excitation/emission of 495 nm/537 nm, from the top of the plate, with 80% gain using a Synergy Neo 2 BioTek plate reader:
First measurement=READING 1
Liquid was removed from wells and fresh 20 mM Tris pH 7.5 without dye was added and plate was read again=READING 2
Liquid was removed from wells and fresh 20 mM Tris pH 7.5 without dye was added and plate was read again=READING 3
Liquid was removed from wells and fresh 20 mM Tris pH 7.5 without dye was added and plate was read again=READING 4
Liquid was removed from wells and fresh 20 mM Tris pH 7.5 without dye was added and plate was read again=READING 5
Liquid was removed from wells and fresh 20 mM Tris pH 7.5 without dye was added and plate was read again=READING 6
Liquid was removed from wells and fresh 20 mM Tris pH 7.5 without dye was added and plate was read again=READING 7
Step 11: To determine the relative DNA adherence to test materials, the last fluorescence readings obtained from the material+dye samples was subtracted from the last fluorescence readings obtained from material+DNA+dye samples. Errors were propagated by quadrature.

### Test Method 2: (Fluorescence)

Samples were measured free standing in the front sample position (sample angled 30 degrees right of normal to incident and detector optics 10 degrees right of normal) on a Perkin Elmer Lambda 1050 spectrophotometer fitted with a PELA 1002 integrating sphere accessory. The scan speed was set to 102 nm/min, the UV-Vis integration was set to 0.56 sec/pt, the data interval was set to 1 nm and the slit width was set to 5 nm. The instrument was set to “% Transmission” and “% Reflectance” mode.

For comparison to a known reference, a 10 ppm quinine solution in 0.5 N sulfuric acid was prepared from quinine hemisulfate monohydrate and presented in a 10 mm quartz cell.

### Test Method 3: (X-ray Photoelectron Spectroscopy)

The sample surfaces were examined using X-ray Photoelectron Spectroscopy (XPS) also known as Electron Spectroscopy for Chemical Analysis (ESCA). This technique provides an analysis of the outermost 3 to 10 nanometers (nm) on the specimen surface. The photoelectron spectra provide information about the elemental and chemical (oxidation state and/or functional group) concentrations present on a solid surface. It is sensitive to all elements in the periodic table except hydrogen and helium with detection limits for most species in the 0.1 to 1 atomic % concentration range. XPS concentrations should be considered semi-quantitative unless standards are included in the data set. Analysis conditions are described in Table 2.

TABLE 2
XPS Measurement Conditions

| | |
|---|---|
| Instrument | Nexsa XPS System, Thermo Scientific |
| Analysis Area | ≈400 μm |
| Photoelectron Take-off Angle | 90° ± 30° solid angle of acceptance |
| X-ray Source | Monochromatic Al Kα (1486.6eV) 72 W |
| Charge Neutralization | Low energy e⁻ and Ar⁺ flood sources |
| Charge Correction | None |
| Analysis Chamber Pressure | <5 × 10⁻⁷ mbar |

### Test Method 4: (Confocal Microscopy)

The samples with fluorescent labels were imaged using a confocal microscope (Zeiss Axioplan 2 with LSM 510 Laser Module, Zeiss, Thornwood N.J.) equipped with an Achroplan 63×/1.4 Oil DIC M27 (FWD=0.19 mm) objective. The film samples were adhered on a 1 in×3 in microscope slide using a droplet of Resolve™ Microscope Immersion Oil (Cornwell Corp., Riverdale, NJ) and covered with a glass cover slip, onto which another droplet of microscope oil was added. The fluorescent images were then taken using 488 laser excitation at 60% power and a 505 nm long pass filter. The scanning parameters were set to define a field of view of 23.88 microns×23.88 microns.

### Test Method 5: (Scanning Electron Microscopy)

Samples were mounted on Aluminum examination stubs and coated with AuPd by DC sputtering to ensure conductivity. Examinations were performed in a Hitachi 54700 Field Emission Scanning Electron Microscope.

### PREPARATORY EXAMPLES

### Preparatory Example 1

An acrylate solution was prepared by first adding 75 wt % PHOTOMER 6210 with 25 wt % SR238 and 0.5% TPO to create Acrylate Resin A. 93 wt % of Acrylate Resin A was added to 7 wt % of HFPO-UA solution, resulting in a second acrylate mixture. Acrylate solution A was then created by manually combining 14 wt % of the second acrylate mixture with 43 wt % PGME and 43 wt % MEK.

### Preparatory Example 2

Resin D was prepared by combining and mixing PHOTOMER 6210, SR238, SR351 and TPO in weight ratios of 60/20/20/0.5.

### Preparatory Example 3: (THV Solution)

Fluoropolymer solution 1 was prepared by adding 5 g of THV220G to a solution of 77.5 g MEK and 17.5 g MIBK.

### Preparatory Example 4: (SiC_{y}/Oₓ coating)

A silicon containing etch resist was deposited onto ST505 film using a home-built parallel plate capacitively coupled plasma reactor as described in U.S. Pat. Nos. 6,696,157). The chamber has a central cylindrical powered electrode with a surface area of 1.7 m² (18.3 ft²). After placing the nanostructured tooling film on the powered electrode, the reactor chamber was pumped down to a base pressure of less than 1.3 Pa (2 mTorr). O₂ and HMDSO gasses were flowed into the chamber at a rate of 2000 SCCM, and 100 SCCM respectively. Treatment was carried out using a plasma enhanced CVD method by coupling RF power into the reactor at a frequency of 13.56 MHz and an applied power of 7500 watts. Treatment time was controlled by moving the film through the reaction zone at rate of 15 ft/min, resulting in an approximate exposure time of 20 seconds. After completing the deposition, RF power was turned off and gasses were evacuated from the reactor. Following the 1st treatment, a 2nd plasma treatment was carried out in the same reactor without returning the chamber to atmospheric pressure. O2 gas was flowed into the chamber at approximately 1000 SCCM. 13.56 MHz RF power was subsequently coupled into the reactor with an applied power of 6000 W. The film was then carried through the reaction zone at a rate of 30 ft/min, resulting in an approximate exposure time of 10 seconds. At the end of this treatment time, the RF power and the gas supply were stopped and the chamber was returned to atmospheric pressure.

### Preparatory Example 5: (THV Coating)

The fluoropolymer solution (5% solids, Fluoropolymer 1 in coating solutions table) was die coated in a roll-to-roll process onto ST505 film with a slot die at a rate of 0.0254 m/s. The solution was coated 10.2 cm wide and pumped with a Harvard syringe pump at a rate of 1.20 sccm. The coating was dried at 37.8° C. for 4 minutes.

### Preparatory Example 6: (Roughened SiCyOx Coating)

A randomly nanostructure silicon containing etch resist was deposited onto Zeoner COP film using a parallel plate capacitively coupled plasma reactor as described in U.S. Pat. Nos. 6,696,157). The chamber has a central cylindrical powered electrode with a surface area of 1.7 m² (18.3 ft²).

After placing the film on the powered electrode, the reactor chamber was pumped down to a base pressure of less than 1.3 Pa (2 mTorr). O2, and HMDSO gasses were flowed into the chamber at a rate 18 SCCM, and 750 SCCM respectively. Treatment was carried out using a plasma enhanced CVD method by coupling RF power into the reactor at a frequency of 13.56 MHz and an applied power of 7500 watts. Treatment time was controlled by moving the film through the reaction zone at rate of 17 ft/min, resulting in an approximate exposure time of 17 seconds.

After completing the deposition, RF power was turned off and gasses were evacuated from the reactor. Following the 1st treatment, a 2nd plasma treatment was carried out in the same reactor without returning the chamber to atmospheric pressure. TMS and O₂ gases were flowed into the chamber at approximately 500 SCCM and 2000 SCCM respectively. 13.56 MHz RF power was subsequently coupled into the reactor with an applied power of 2000 W. The film was then carried through the reaction zone at a rate of 30 ft/min, resulting in an approximate exposure time of 10 seconds.

Following the 2nd treatment, a 3rd plasma treatment was carried out in the same reactor without returning the chamber to atmospheric pressure. O₂ gas was flowed into the chamber at approximately 2000 SCCM. 13.56 MHz RF power was subsequently coupled into the reactor with an applied power of 2000 W. The film was then carried through the reaction zone at a rate of 30 ft/min, resulting in an approximate exposure time of 10 seconds. At the end of this treatment time, the RF power and the gas supply were stopped and the chamber was returned to atmospheric pressure.

### EXAMPLES

### Example 1: (Wells)

Step 1: A nano-featured template film was prepared by die coating Resin D onto a polycarbonate film. The coated film was pressed against a nanostructured nickel surface attached to a steel roller controlled at 60° C. using a rubber covered roller at a speed of 15.2 meters/min. The nanostructured nickel tool consists of twelve 6 mm by 6 mm patterned areas with features ranging in size between 75 nm and 500 nm. The patterned area consisted of a multi-pitch pattern with pitches of 150, 200 and 250 nm with feature widths of half the pitch (75, 100, 125 nm). The features were arranged in a square grid so that pitches were varied in both axes resulting in a nine unit repeating cell with rectangles of all combinations of widths mentioned above. In this repeating cell, the 150 nm pitch sections had 27 features, the 200 nm pitch sections had 20 features and the 250 nm pitch sections had 16 features. The features were about 200 nm tall and had side wall angles of approximately 4 degrees. The coating thickness of Resin D on the film was sufficient to fully wet the nickel surface and form a rolling bead of resin as the coated film was pressed against the nanostructured nickel surface. The film was exposed to radiation from two Fusion UV lamp systems (obtained under the trade designation “F600” from Fusion UV Systems, Gaithersburg, MD) fitted with D bulbs both operating at 142 W/cm while in contact with the nanostructured nickel surface. After peeling the film from the nanostructured nickel surface, the nanostructured side of the film was exposed again to radiation from the Fusion UV lamp system.
Step 2: A silicon containing release film layer assembled according to methods described in U.S. Pat. No. 6,696,157 (David et al.) and U.S. Pat. No. 8,664,323 (Iyer et al.) and U.S. Patent Publication No. 2013/0229378 (Iyer et al.) was applied to the nanostructure tooling film in a parallel plate capacitively coupled plasma reactor. The chamber has a central cylindrical powered electrode with a surface area of 1.7 m² (18.3 ft²). After placing the nanostructured tooling film on the powered electrode, the reactor chamber was pumped down to a base pressure of less than 1.3 Pa (2 mTorr). O₂ gas was flowed into the chamber at a rate of 1000 SCCM. Treatment was carried out using a plasma enhanced CVD method by coupling RF power into the reactor at a frequency of 13.56 MHz and an applied power of 2000 watts. Treatment time was controlled by moving the nanostructured tooling film through the reaction zone at rate of 9.1 meter/min (30 ft/min) resulting in an approximate exposure time of 10 seconds. After completing the deposition, RF power was turned off and gasses were evacuated from the reactor. Following the 1st treatment, a 2nd plasma treatment was carried out in the same reactor without returning the chamber to atmospheric pressure. HMDSO gas was flowed into the chamber at approximately 1750 SCCM to achieve a pressure of 9 mTorr. 13.56 MHz RF power was subsequently coupled into the reactor with an applied power of 1000 W. The film was then carried through the reaction zone at a rate of 9.1 meter/min (30 ft/min) resulting in an approximate exposure time of 10 seconds. At the end of this treatment time, the RF power and the gas supply were stopped, and the chamber was returned to atmospheric pressure.
Step 3: A silicon containing etch resist was deposited onto ST505 film according to Preparatory Example 4.
Step 4: Tie Layer 1 (0.3% Bis-3-trimenthoxy silyl propylamine (Silquest A-1170) in methyl ethyl ketone) was die coated in a roll-to-roll process onto the silicon containing etch resist from step 3 at a rate of 0.1 m/s using a slot die. The solution was coated 15.24 cm wide and pumped with a Harvard syringe pump at a rate of 4 sccm. The coating was dried at 65° C. for 1 minute.
Step 5: Fluoropolymer Solution 1 was die coated in a roll-to-roll process onto the substrate from step 4 with a slot die at a rate of 0.0254 m/s. The solution was coated 10.2 cm wide and pumped with a Harvard syringe pump at a rate of 1.20 sccm. The coating was dried at 65° C. for 4 minutes.
Step 6: Tie layer 2 was die coated in a roll-to-roll process onto the film from step 5 at a rate of 0.05 m/s using a slot die. The solution was coated 10.2 cm wide and pumped with a Harvard syringe pump at a rate of 1.75 sccm. The coating was dried at 65° Cs for 2 minutes.
Step 7: The release treated template film created in Step 2 was slot-die coated with a solution of Acrylate solution A at 0.05 meters per second. The solution was coated 10.16 cm wide and pumped with a Harvard syringe pump at a rate of 0.90 sccm. The coating was partially cured 12 meters from the solution application using a 405 nm UV-LED system powered at 0.5 Amps at volts. The film entering a nip 2 meters thereafter. At the nip, the film with the silicon containing layer and the fluorinated layer was laminated with the overcoated release treated template film. The nip consisted of a 90-durometer rubber roll and a steel roll set at 54° C. The nip was engaged by two Bimba air cylinders pressed by 0.55 MPa.

The solution was cured using a Fusion D bulb and the acrylate mixture was separated from the release treated template film remaining on the silicon and fluorine containing film for the entirety of the 6 mm by 6 mm patterned areas to create a masked nano-featured film. Web tensions were set to be approximately 0.0057 N/mm.
Step 8: Following the low-land transfer process, reactive ion etching was carried out on the patterned film in the same home-built reactor chamber used to deposit the PECVD release layer. After placing the coated film on the powered electrode, the reactor chamber was pumped down to a base pressure of less than 1.3 Pa (1 mTorr). A mixture of C₆F₁₄ and O₂ gas was flowed into the chamber at a rate of 100 SCCM and 50 SCCM, respectively. 13.56 MHz RF power was subsequently coupled into the reactor with an applied power of 7500 W. The film was then carried through the reaction zone at a rate of 4.5 ft/min, to achieve an exposure time of approximately 67 sec in order to transfer the pattern into the top silicon containing etch resist. After completing the 1st etch step, RF power was turned off and gasses were evacuated from the reactor. Following the 1st etch, a 2nd reactive ion etching treatment was carried out in the same reactor without returning the chamber to atmospheric pressure. C₆F₁₄ gas was flowed into the chamber at a flow rate of 100 SCCM. 13.56 MHz RF power was subsequently coupled into the reactor with an applied power of 7500 W. The nanostructured film was then carried through the reaction zone at a rate of 20 ft/min, resulting in an approximate exposure time of 15 seconds. At the end of this treatment time, the RF power and the gas supply were stopped and the chamber was returned to atmospheric pressure.

### Example 2: (Posts)

Step 1: Well tooling film was created by feeding Acrylate Resin A into a nip at 3-4 cc/min into a lamination nip to maintain a width of approximately 12-15 cm. The two films entering the nip where the film from Step 2 of Example 1 and Melenex 454. The process was run at 3.0 m/min (10 fpm) with nip pressures on the two Bimba air cylinders set to 0.28 MPa (40 psi) and the temperature of the lamination roll set to 65.6° C. (150° F.).
Step 2: A release layer was deposited on the tooling film by the process described in Step 2 of Example 1.
Step 3: The fluoropolymer solution (5% solids, Fluoropolymer 1 in coating solutions table) was die coated in a roll-to-roll process onto COP film with a slot die at a rate of 0.0254 m/s. The solution was coated 10.2 cm wide and pumped with a Harvard syringe pump at a rate of 1.20 sccm. The coating was dried at 37.8° C. for 4 minutes.
Step 4: A silicon containing etch resist was deposited onto the THV220G/COP film as described in Step 3 of Example 1
Step 5: Tie layer 3 was die coated in a roll-to-roll process onto the film from Step 4 at a rate of 0.10 m/s using a slot die. The solution was coated 15.2 cm wide and pumped with a Harvard syringe pump at a rate of 3 sccm. The coating was dried in an oven set to 93.3° C. for 1 minute.
Step 6: Resin A was coated onto the release treated tooling film from Step 2, and then laminated with the film from Step 5, to transfer nanostructured posts of Resin A, according to the procedure described in Step 7 of Example 1, to create a masked nano-featured film.
Step 7: Following the transfer process, reactive ion etching was carried out on the masked nano-featured film in the same home-built reactor chamber used to deposit the PECVD release layer. After placing the coated film on the powered electrode, the reactor chamber was pumped down to a base pressure of less than 1.3 Pa (1 mTorr). A mixture of C₆F₁₄ and O₂ gas was flowed into the chamber at a rate of 100 SCCM and 50 SCCM, respectively. 13.56 MHz RF power was subsequently coupled into the reactor with an applied power of 7500 W. The film was then carried through the reaction zone at a rate of 0.46 m/min (1.5 ft/min), to achieve an exposure time of 200 sec in order to transfer the pattern into the top silicon containing etch resist. After completing this first etch step, RF power was turned off and gasses were evacuated from the reactor.
Step 8: Following the first etch, a second reactive ion etching treatment was carried out in the same reactor without returning the chamber to atmospheric pressure. O₂ gas was flowed into the chamber at a flow rate of 275 SCCM. 13.56 MHz RF power was subsequently coupled into the reactor with an applied power of 7500 W. The film was then carried through the reaction zone at a rate of 1.8 m/min (6 ft/min). At the end of this treatment time, the RF power and the gas supply were stopped and the chamber was returned to atmospheric pressure.

Photograph of the post construction are shown in FIGS. 7A-7B.

### Example 3: (Posts on COP Film)

Step 1: A release treated tooling film was made according to Example 2 Steps 1 and 2.
Step 2: A silicon containing etch resist was deposited onto COP film as described in Step 3 of Example 1.
Step 3: Tie layer 3 was coated onto the silicon containing etch resist/COP film as described in Step 5 from Example 2.
Step 4: Resin A was coated onto the release treated tooling film from Step 1, and then laminated with the film from Step 3, to transfer nanostructured posts of Resin A, according to the procedure described in Step 7 of Example 1, to create a masked nano-featured film.
Step 5: A first etch was performed according to Step 7 of Example 2 to break through the silicon containing etch resist.
Step 6: Following the first etch, a second reactive ion etching treatment was carried out in the same reactor without returning the chamber to atmospheric pressure. O₂ gas was flowed into the chamber at a flow rate of 300 SCCM. 13.56 MHz RF power was subsequently coupled into the reactor with an applied power of 7500 W. The film was then carried through the reaction zone at a rate of 2.4 m/min (8 ft/min). At the end of this treatment time, the RF power and the gas supply were stopped and the chamber was returned to atmospheric pressure.

Photographs of the post construction are shown in FIGS. 7C-7D.

### Example 4: (DNA Adherence)

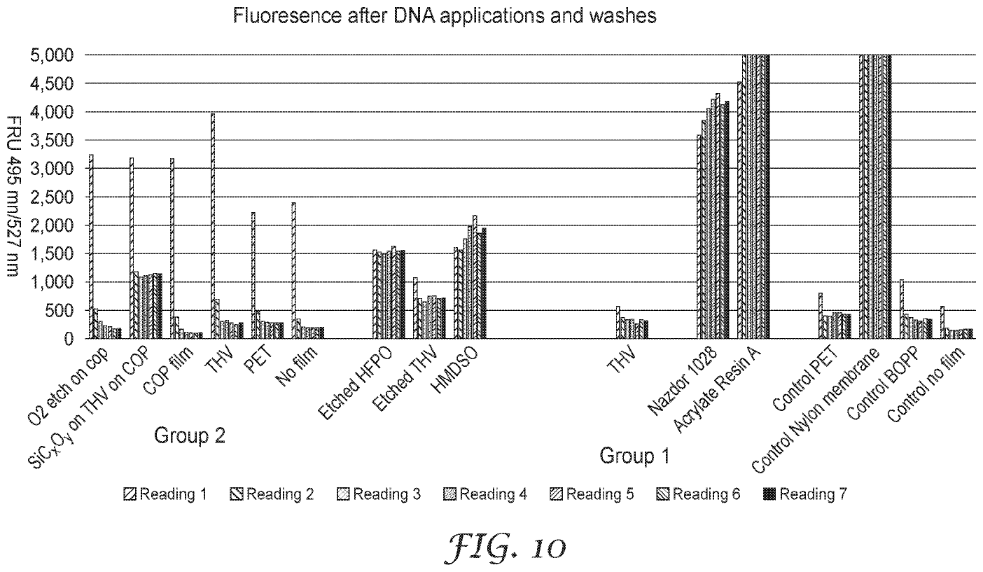

DNA adherence was measured according to Test Method 1, and the results are shown in Table 3 below, and plotted in FIG. 10. Image analysis was used to quantify the intensity of fluorescence images (units are arbitrary).

TABLE 3
Results of DNA Adherence Tests

| | (Material + Dye + | |
|---|---|---|
| | DNA) − | |
| | (Material + Dye) | Standard |
| Material | Image Brightness | Deviation |
| ST505 release treated | 1,971 | 2,326 |
| according to Example 1, | | |
| step 2 | | |
| THV on COP and etch resist | 41 | 42 |
| according to Example 1, | | |
| steps 3-5 | | |
| ST505 | 74 | 55 |
| ST505 coated with Acrylate | 5,016 | 1,245 |
| solution A according to | | |
| Example 1, step 7 | | |
| Silicon containing etch | 5 | 73 |
| resist of Example 1, | | |
| Step 3 on COP | | |
| COP | −10 | 18 |
| Control (no film) | −34 | 29 |

### Example 5 (Fluorescence)

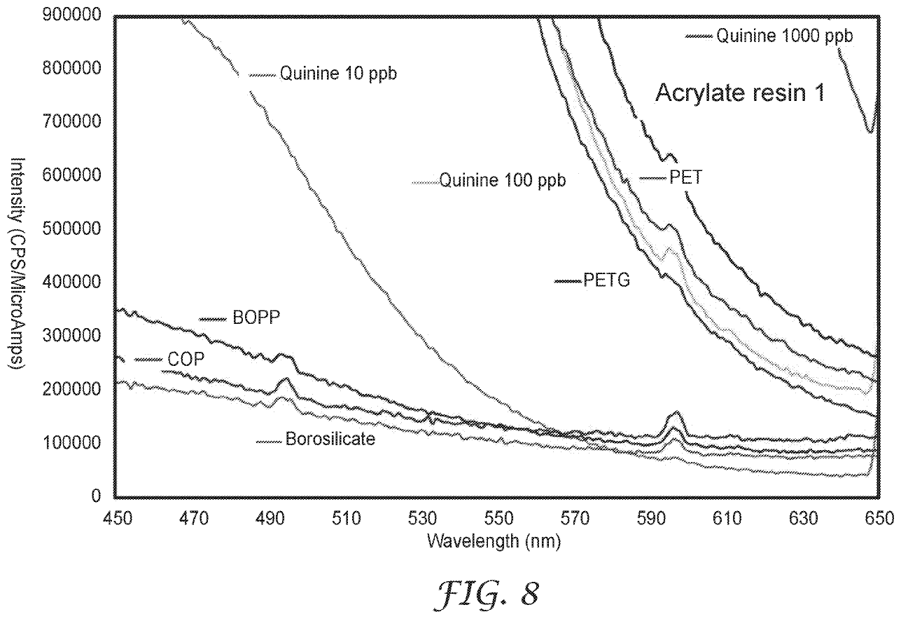

Fluorescence was measured according to Test Method 2. The intensity in CPS/MicroAmps is shown in Table 4 below, and the results are plotted in FIG. 8.

TABLE 4
Results of Fluorescence Tests

| Wavelength | | | | | Quinine |
|---|---|---|---|---|---|
| (nm) | Borosilicate | COP | ST505 | BOPP | 10 ppb |
| 480 | 183759.0 | 212657.8 | 9425370.00 | 284921.2 | 822324.9 |
| 490 | 168956.9 | 193708.6 | 6895010.00 | 259417.8 | 695519.1 |
| 500 | 157828.0 | 183641.9 | 5126270.00 | 230640 | 600745.7 |
| 510 | 145817.8 | 172660.3 | 3787185.00 | 210629 | 484690.4 |

### Example 6: (Silane Coating on Film Surfaces)

A silane coating solution was prepared by mixing 3-aminopropyltrimethoxysilane (1.00 g), absolute ethanol (46.3 g), acetic acid (200 mg), and water (2.5 g). The coated films of Preparatory Examples 4 and 5 were cut into 4 inch squares and immersed in silane coating solution for 45 minutes. Immediately after removing from the solution, the films were rinsed with excess ethanol using a squirt bottle, then placed in an oven held at 70° C. for 30 minutes. Films were then analyzed by XPS before and after silane treatment to determine the relative concentrations of nitrogen on the surfaces. Results are shown in Table 5.

TABLE 5
Results of XPS Tests

| Film | Percent Nitrogen by XPS |
|---|---|
| Preparatory Example 4 | 0.1% |
| Preparatory Example 4, silane-treated | 1.4% |
| Preparatory Example 5 | 0.2% |
| Preparatory Example 5, silane-treated | 0.1% |

### Example (Peeled Wells)

TABLE 6
Materials for Example 7

| Abbreviation | Description and Source |
|---|---|
| Coating Solution 1 | 0.25 wt % PVA in a 75/25 solution of |
| | IPA/H2O with 0.025% Tergitol 15-S-7 |
| Coating Solution 2 | 2.5 wt % PVB 30H in IPA |
| Fluoropolymer Solution 2 | 1 wt % THV221A in a 80/20 solution of |
| | MEK/MIBK |
| Acrylate Resin A | 75 wt % Photomer 6210 with 25 wt % |
| | SR238 and 0.5% TPO |
| PVA | 9,000-10,000 Molecular Weight, 80% |
| | hydrolized polyvinyl alcohol, Sigma- |
| | Aldrich Inc., St Louis, MO |
| PVB 30 H | Mohwitol ® Polyvinyl butyral 30 H, |
| | Kuraray America Inc, Tokyo, Japan |
| Tergitol ™ | TergitolTM 15-S-7, Sigma-Aldrich Inc, St |
| | Louis, MO |
| IPA | Isopropyl Alcohol; Brenntag Great Lakes, |
| | Wauwatosa, WI |
| MEK | Methyl ethyl ketone; Brenntag Great |
| | Lakes, Wauwatosa, WI |
| MIBK | Methyl isobutyl ketone; Brenntag Great |
| | Lakes, Wauwatosa, WI |
| TPO | Diphenyl(2,4,6-trimethylbenzoyl) |
| | phosphine oxide, IRGACURE TPO; |
| | BASF, Florham Park, NJ |
| THV221A | THV, Dyneon THV 221A; 3M Co, Saint |
| | Paul, MN |

Step 1: A nano-featured template film was prepared by die coating Resin D onto a polycarbonate film. The coated film was pressed against a nanostructured nickel surface attached to a steel roller controlled at 60° C. using a rubber covered roller at a speed of 15.2 meters/min. The nanostructured nickel tool consists of a 90 mm×90 mm square of 275 nm diameter wells at a pitch of 600 nm. The features were about 250 nm deep and had side wall angles of approximately 2 degrees. The coating thickness of Resin D on the film was sufficient to fully wet the nickel surface and form a rolling bead of resin as the coated film was pressed against the nanostructured nickel surface. The film was exposed to radiation from two Fusion UV lamp systems (obtained under the trade designation “F600” from Fusion UV Systems, Gaithersburg, MD) fitted with D bulbs both operating at 142 W/cm while in contact with the nanostructured nickel surface. After peeling the film from the nanostructured nickel surface, the nanostructured side of the film was exposed again to radiation from the Fusion UV lamp system.
Step 2: A silicon containing release film layer assembled according to methods described in U.S. Pat. No. 6,696,157 (David et al.) and U.S. Pat. No. 8,664,323 (Iyer et al.) and U.S. Patent Publication No. 2013/0229378 (Iyer et al.) was applied to the nanostructure tooling film in a parallel plate capacitively coupled plasma reactor. The chamber has a central cylindrical powered electrode with a surface area of 1.7 m2 (18.3 ft2). After placing the nanostructured tooling film on the powered electrode, the reactor chamber was pumped down to a base pressure of less than 1.3 Pa (2 mTorr). O2 gas was flowed into the chamber at a rate of 1000 SCCM. Treatment was carried out using a plasma enhanced CVD method by coupling RF power into the reactor at a frequency of 13.56 MHz and an applied power of 2000 watts. Treatment time was controlled by moving the nanostructured tooling film through the reaction zone at rate of 9.1 meter/min (30 ft/min) resulting in an approximate exposure time of 10 seconds. After completing the deposition, RF power was turned off and gasses were evacuated from the reactor. Following the 1st treatment, a 2nd plasma treatment was carried out in the same reactor without returning the chamber to atmospheric pressure. HMDSO gas was flowed into the chamber at approximately 1750 SCCM to achieve a pressure of 9 mTorr. 13.56 MHz RF power was subsequently coupled into the reactor with an applied power of 1000 W. The film was then carried through the reaction zone at a rate of 9.1 meter/min (30 ft/min) resulting in an approximate exposure time of 10 seconds. At the end of this treatment time, the RF power and the gas supply were stopped, and the chamber was returned to atmospheric pressure.
Step 3: A silicon containing etch resist was deposited onto COP according to Preparatory Example 6.
Step 4: Fluoropolymer Solution 2 was die coated in a roll-to-roll process onto the substrate from step 4 with a slot die at a rate of 0.0381 m/s. The solution was coated 15.24 cm wide and pumped with a Harvard syringe pump at a rate of 1.9 sccm. The coating was dried at 65° C. for 3 minutes.
Step 5: Coating Solution 1 was die coated in a roll-to-roll process onto the film from step 5 at a rate of 0.05 m/s using a slot die. The solution was coated 15.24 cm wide and pumped with a Harvard syringe pump at a rate of 6.6 sccm. The coating was dried at 65° C. for 2 minutes.
Step 6: The release treated template film created in Step 2 was slot-die coated with a solution of Coating solution 2 at 0.05 meters per second. The solution was coated 15.24 cm wide and pumped with a Harvard syringe pump at a rate of 4.5 sccm. The coating was dried in a gap drier 1 m after coating. The film entered a nip 14 meters thereafter. At the nip, the film with the silicon containing layer and the fluorinated layer was laminated with the overcoated release treated template film. The nip consisted of a 90-durometer rubber roll and a steel roll set at 65° C. The nip was engaged by two Bimba air cylinders pressed by 0.55 MPa.

The Coating Solution 2 was separated from the release treated template film remaining on the silicon and fluorine containing film for the entirety of the 8.9 cm by 8.9 cm patterned areas to create a masked nano-featured film. Web tensions were set to be approximately 0.0057 N/mm
Step 7: Following the low-land transfer process, reactive ion etching was carried out on the patterned film in the same home-built reactor chamber used to deposit the PECVD release layer. After placing the coated film on the powered electrode, the reactor chamber was pumped down to a base pressure of less than 1.3 Pa (1 mTorr). O2 gas was flowed into the chamber at a rate of 1000 SCCM. 13.56 MHz RF power was subsequently coupled into the reactor with an applied power of 4000 W. The film was then carried through the reaction zone at a rate of 5 ft/min, to achieve an exposure time of approximately 60 sec in order to transfer the pattern into the fluoropolymer layer. After completing the 1st etch step, RF power was turned off and gasses were evacuated from the reactor and the chamber was returned to atmospheric pressure.
Step 8: The etched layers of Coating solution 2 and Coating Solution 1 were peeled off of the Fluoropolyer Solution 2 layer. Acrylate Resin A was dispensed on the etched pattern film with a syringe pump and a sheet of roughed ST505 was laminated over the top such that the entire 8.9 cm by 8.9 cm patterned area was covered with Acrylate Resin A. Acrylate Resin A was then cured while using a D bulb at room temperature, 20° C. Then the ST505 layer was peeled from the etched nanostructure film. The Coating solution 2 and Coating Solution 1 layers peeled off the etched nanostructure film, leaving an unetched fluoropolymer layer with 250 nm wells where the bottom of the wells is the structured SiCOx layer.

Scanning electron micrographs of the peeled wells of Example 7 were obtained using Test Method 5 (Scanning Electron Microscopy) as described above. The SEM micrographs show a peeled material stack with rough SiCOx at the base of the features and THV in the interstitial spaces.

### Example 8: (Fluorescent Labeling of Aminosilane-Treated Wells)

Example 7 was cut into 1.5 cm squares and placed in a 12 well plate. An amino silane coating solution was prepared by vortex mixing 3-aminopropyltrimethoxysilane (0.4 g), absolute ethanol (18.52 g), acetic acid (80 μl) and deionized water (1.0 g) in a 25 mL glass vial. Then, 2 g of the amino silane solution was injected into each well containing a 1.5 cm-square well sample. The well plate was allowed to stir in a low-speed orbital shaker, which was set at 60 rpm, for 1 hr. The patterned samples were rinsed with ethanol three times, dried with nitrogen and placed in an oven held at 70° C. for 30 minutes.

For the fluorescent labeling, the films were placed in a 12 well plate and rinsed with TE buffer pH 8.0 for three times. Approximately 500 μL of a 0.1 mg/mL Alexa Fluor™ 488 NHS ester (succinimidyl ester) in TE buffer pH 8.0 was pipeted onto the surface of the aminosilane-functionalized well samples. The functionalization was set for an hour then the samples were rinsed with TE buffer pH 8.0, dried with nitrogen and imaged using a confocal microscope.

Confocal images of the fluorescent nanowell construction of Example 8 were obtained using Test Method 4 (Confocal Microscopy) as described above. The confocal micrographs show that we have achieved selectivity/contrast in the fluorescence of the bottoms of the wells versus the tops with increased fluorescence intensity in the wells then in the interstitial regions.

### PROPHETIC EXAMPLES

### Prophetic Example 1 (Wells Method 1—Etch)

Step 1: A masked nano-featured film may be prepared following the procedure described in Steps 1-7 of Example 1, where COP film can be used in place of ST505 in Step 3 of Example 1.
Step 2: The mask and anti-biofouling layers may be etched to expose the inorganic layer at the bottoms of the features, and the anti-biofouling layer at the top plane. The final etch conditions may be chosen such that the top plane remains anti-biofouling and resists alkoxy silane binding, and the bottom of the wells can be functionalized using alkoxy silanes.
Step 3: A functional alkoxy silane such as aminopropyltrimethoxysilane may be bound to the silicon oxide material by solution coating and optionally rinsing. This silane does not react with the THV surface. The silane may contain a reactive group that can be used to form a hydrogel polymer in the wells. The alkoxysilane may contain an acrylamide group. After well functionalization, acrylamide is polymerized on the surface, leading to growth of poly(acrylamide) in the wells.
Step 4: The flexible substrate may be attached to a rigid backing such as glass or quartz by coating 1-50 um of 3M™ Optically Clear Adhesive 8171, Adhesive A, Adhesive B, or Adhesive C, onto the flexible substrate then laminating the flexible substrate to the rigid backing.

### Prophetic Example 2 (Wells Method 2—Peel)

Step 1: A tooling film with nanostructured holes may be prepared as in Steps 1 and 2 of Example 1.
Step 2: COP film may be coated with an inorganic layer and a tie layer as described in
Steps 3 and 4 of Example 1. A fluoropolymer layer may be coated as described in Step 5 of Example 1, however, the flow rate and concentration of the fluoropolymer may be adjusted so the dry thickness is 10-100 nm thick.
Step 3: A 10-200 nm thick PVA layer may be die coated on the THV layer. The PVA may be dissolved in a mixture of water and IPA, and a surfactant such as Tergitol 15-S-7 (DowDuPont, Midland, MI) may be added to aid spreading and coating.
Step 4: A structured pattern of Acrylate Resin A holes may be transferred onto the PVA layer using the tooling film from Step 1 as described in Step 7 of Example 1.
Step 5: The silicon containing layer may be exposed using a plasma etch as described in Step 8 of Example 1.
Step 6: The PVA may be removed from the top layer of fluoropolymer by either washing with water under an ultrasonic horn, or by coating the PVA with 1-5 um of Acrylate Resin A, laminating to a PET film, then curing and peeling the PVA off the fluoropolymer onto the PET film.
Step 7: A functional alkoxy silane may be bound to the silicon oxide material as described in Step 3 of Prophetic Example 1
Step 8: The flexible substrate may be attached to a rigid backing as described in Step 4 of Prophetic Example 1

### Prophetic Example 3 (Posts Method 1—Etch)

Step 1: A masked nano-featured film is prepared following the procedure described in Steps 1-7 of Example 2.
Step 2: The mask and anti-biofouling layers are etched to expose the anti-biofouling layer at the bottom plane, and the inorganic layer at to the top of the posts. The final etch conditions are chosen such that the bottom plane remains anti-biofouling and resists alkoxy silane binding, and the top of the posts can be functionalized using alkoxy silanes.
Step 3: A functional alkoxy silane such as aminopropyltrimethoxysilane may be bound to the silicon oxide material by solution coating. This silane does not react with the THV surface. The silane contains a reactive group that can be used to form a hydrogel polymer on the posts. The alkoxysilane may contain an acrylamide group. After post functionalization, acrylamide is polymerized on the surface, leading to growth of poly(acrylamide) on the posts.
Step 4: The flexible substrate may be attached to a rigid backing as described in Step 4 of Prophetic Example 1.

### EMBODIMENTS

Embodiment A. An article, comprising:
a flexible carrier film with a first major surface and a second major surface, wherein a first major surface of the flexible carrier film comprises an array of structures extending away therefrom, wherein at least a portion of the structures comprise:
an inorganic layer with a first major surface and a second major surface, wherein the first major surface of the inorganic layer is on the flexible carrier film,
an analyte binding layer with a first major surface on the second major surface of the inorganic layer, wherein the analyte binding is bonded to the inorganic layer via a network of hydrocarbon linking groups, and wherein the second major surface of the analyte binding layer comprises at least one functional group selected to bind with a biochemical analyte; and
recessed features interspersed with the structures, wherein the recessed features are free of the inorganic layer and the analyte binding layer.

Embodiment B. The article of Embodiment A, wherein the flexible carrier film comprises a polymeric material chosen from cyclic olefin polymer (COP), biaxially oriented polypropylene (BOPP), poly(meth)acrylates and copolymers, polyamides, polyesters, polycarbonates, hydrogenated styrenics, and combinations thereof.

Embodiment C. The article of Embodiments A or B, wherein the flexible carrier film further comprises an anti-biofouling layer.

Embodiment D. The article of Embodiment C, wherein the anti-biofouling layer comprises a material chosen from fluoropolymers, non-aromatic hydrocarbon polymers, cyclic olefin polymers, cyclic olefin copolymers, cyclic block copolymers, silicones, metals, a methyl terminated layer, a noble metal and mixtures and combinations thereof.

Embodiment E. The article of Embodiment D, wherein the anti-biofouling layer comprises a fluoropolymer, a cyclic olefin copolymer or a methyl terminated layer.

Embodiment F. The article of any of Embodiments A to E, wherein the inorganic layer has a thickness of less than 100 nm.

Embodiment G. The article of any of Embodiments A to F, wherein the inorganic layer comprises an oxide of Si, Ti or Al.

Embodiment H. The article of Embodiment G, wherein the oxide is chosen from SiO₂, SiCₓO_{y}, SiAlₓO_{y}, TiO, AlOₓ and mixtures and combinations thereof.

Embodiment I. The article of any of Embodiments A to H, wherein the analyte binding layer is chosen from a reactive silane, a functionalizable hydrogel, a functionalizable polymer, and mixtures and combinations thereof.

Embodiment J. The article of Embodiment I, wherein the analyte binding layer comprises acrylamide copolymers, condensed silanes, and mixtures and combinations thereof.

Embodiment K. The article of Embodiment J, wherein the hydrocarbon linking group is at least one methylene unit long, and wherein the hydrocarbon linking group and can be linear, cyclic, branched, or aromatic.

Embodiment L. The article of Embodiment K, wherein the hydrocarbon linking group includes heteroatoms.

Embodiment M. The article of Embodiment L, wherein the hydrocarbon linking group is derived from the condensation of a functional silane onto the inorganic layer.

Embodiment N. The article of Embodiment M, wherein the functional silane comprises functional groups chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, or azos and mixtures and combinations thereof.

Embodiment O. The article of Embodiment N, wherein the functional group is chosen from alkene, azide, amino, carboxylic acid, hydrazone, halogen, hydroxy, tetrazole, tetrazinc, thiol, and combinations thereof.

Embodiment P. The article of any of Embodiments A to O, wherein the structures have a height above the first major surface of the flexible carrier film of greater than 0 nm and less than 1000 nm.

Embodiment Q. The article of Embodiment P, wherein the structures have a heights of 20 nm to 200 nm.

Embodiment R. The article of any of Embodiments A to Q, wherein the structures comprise posts with a diameter of 10 nm to 10,000 nm.

Embodiment S. The article of Embodiment R, wherein the posts have an aspect ratio (height:diameter) of 5:1 to 1:70.

Embodiment T. The article of any of Embodiments A to S, further comprising an adhesive layer on the second major surface of the flexible carrier film.

Embodiment U. The article of Embodiment T, further comprising a support layer on the adhesive layer, wherein the support layer is chosen from a release liner and a rigid substrate.

Embodiment V. The article of Embodiment U, wherein the rigid substrate is chosen from silicon, glass, plastic, metal, metal oxide, paper, and combinations thereof.

Embodiment W. The article of any of Embodiments A to V, wherein the analyte comprises a biomolecule chosen from amino acids, nucleosides, nucleotides, peptides, oligonucleotides, polynucleotides, nucleic acids, and proteins, carbohydrates, secondary metabolites, pharmaceutical molecules and combinations thereof.

Embodiment X. The article of Embodiment W, wherein the biomolecule is chosen from polynucleotides, oligonucleotides and nucleic acids.

Embodiment Y. The article of any of Embodiments A to X, wherein the second major surface of the inorganic layer is structured.

Embodiment Z. An article, comprising:
a flexible carrier film with a first major surface and a second major surface;
an inorganic layer with a first major surface and a second major surface, wherein the first major surface of the inorganic layer is on the first major surface of the flexible polymeric film;
an anti-biofouling layer on at least a portion of the inorganic layer, wherein the anti-biofouling layer comprises an arrangement of wells, wherein at least a portion of the wells comprise a floor having thereon a first major surface of an analyte binding layer bound to the second major surface of the inorganic layer via a network of hydrocarbon linking groups, and wherein a first major surface of the analyte binding layer in the well comprises at least one functional group reactive with the analyte in the sample fluid; and
structures interspersed with the wells, wherein the structures are free of the analyte binding layer and the inorganic layer.

Embodiment AA. The article of Embodiment Z, wherein the flexible polymeric carrier comprises a polymeric material chosen from cyclic olefin polymer (COP), biaxially oriented polypropylene (BOPP), poly(meth)acrylates and copolymers, polyamides, polyesters, polycarbonates, hydrogenated styrenics, and combinations thereof.

Embodiment BB. The article of Embodiments Z or AA, The article of claim 26, wherein the anti-biofouling layer comprises a material chosen from fluoropolymers, non-aromatic hydrocarbon polymers, cyclic olefin polymers, cyclic olefin copolymers, cyclic block copolymers, silicones, metals, a methyl terminated layer, a noble metal and mixtures and combinations thereof.

Embodiment CC. The article of any of Embodiments Z to BB, wherein the inorganic layer comprises an oxide of Si, Ti or Al.

Embodiment DD. The article of Embodiment CC, wherein the oxide is chosen from SiO₂, SiCₓO_{y}, SiAlₓO_{y}, TiO, AlOₓ and mixtures and combinations thereof.

Embodiment EE. The article of any of Embodiments Z to DD, wherein the analyte binding layer is chosen from a reactive silane, a functionalizable hydrogel, a functionalizable polymer, and mixtures and combinations thereof.

Embodiment FF. The article of Embodiment EE, wherein the analyte binding layer comprises acrylamide copolymers, condensed silanes, and mixtures and combinations thereof.

Embodiment GG. The article of any of Embodiments Z to FF, wherein the hydrocarbon linking group is a methylene group at least one methylene unit long, and wherein the hydrocarbon linking group is linear, cyclic, branched or aromatic.

Embodiment HH. The article of embodiment GG, wherein the hydrocarbon linking group includes heteroatoms.

Embodiment II. The article of Embodiments GG or GH, wherein the hydrocarbon linking group is derived from a reaction with a functional silane having functional groups chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, azos, and mixtures and combinations thereof.

Embodiment JJ. The article of Embodiment II, wherein the functional group is chosen from alkene, azide, amino, carboxylic acid, hydrazone, halogen, hydroxy, tetrazole, tetrazine, thiol, norbornene and combinations thereof.

Embodiment KK. The article of any of Embodiments Z to JJ, wherein the wells comprise walls with a height above the second major surface of the inorganic layer of greater than 0 nm and less than 1000 nm.

Embodiment LL. The article of any of Embodiments Z to KK, wherein the wells have a diameter of 10 nm to 10,000 nm.

Embodiment MM. The article of any of Embodiments Z to LL, further comprising an adhesive layer on the second major surface of the flexible carrier film.

Embodiment NN. The article of Embodiment MM, further comprising a support layer on the adhesive layer, wherein the support layer is chosen from a release liner and a rigid substrate chosen from silicon, glass, plastic, metal, metal oxide, paper, and combinations thereof

Embodiment OO. The article of any of Embodiments Z to NN, wherein the analyte comprises a biomolecule chosen from amino acids, nucleosides, nucleotides, peptides, oligonucleotides, polynucleotides, nucleic acids, and proteins, carbohydrates, secondary metabolites, pharmaceutical molecules and combinations thereof.

Embodiment PP. The article of Embodiment OO, wherein the biomolecule is chosen from polynucleotides, oligonucleotides and nucleic acids.

Embodiment QQ. A method for making a component of a diagnostic device, the method comprising:
depositing an inorganic layer on a first major surface of a flexible carrier film;
providing a patterned layer with a patterned surface including a first arrangement of recessed features, each recessed feature adjoining at least one plateau feature;
covering the patterned surface with a masking layer;
overlaying and contacting the inorganic layer on the flexible carrier film to the masking layer;
removing the patterned layer from the masking layer such that least a portion of the masking layer remains in contact with the inorganic layer, the masking layer comprising an exposed surface with a second arrangement of recessed features and plateau features, wherein the second arrangement of recessed features in the masking layer is an inverse of the first arrangement of recessed features in the patterned layer;
etching the recessed features of the masking layer to remove portions of the inorganic layer between the plateau features thereof to form an arrangement of structures extending away from the first major surface of the flexible carrier film, wherein at least a portion of the structures comprise the inorganic layer on an exposed surface thereof; and
selectively applying a functional silane material on the inorganic layers on the exposed surfaces of the structures such that the functional silane material bonds to the inorganic layer via a network of methylene groups to form an analyte binding layer thereon, wherein the analyte binding layer comprises at least one functional group reactive with a biochemical analyte.

Embodiment RR. The method of Embodiment QQ, further comprising an anti-biofouling layer on the flexible carrier film.

Embodiment SS. The method of Embodiments QQ or RR, further comprising depositing a silane tie layer on the inorganic layer on the flexible carrier film.

Embodiment TT. The method of any of Embodiments QQ to SS, further comprising depositing a silane tie layer on the masking layer.

Embodiment UU. The method of any of Embodiments QQ to TT, wherein the masking layer comprises a (meth)acrylate.

Embodiment VV. The method of Embodiment UU, wherein the masking layer is a UV-curable acrylate.

Embodiment WW. The method of any of Embodiments QQ to VV, wherein the etching comprises a first etch with a fluorine compound and a second etch with an oxygen compound.

Embodiment XX. The method of any of Embodiments QQ to WW, wherein the flexible carrier film comprises a polymeric material chosen from cyclic olefin polymer (COP), biaxially oriented polypropylene (BOPP), poly(meth)acrylates and copolymers, polyamides, polyesters, polycarbonates, hydrogenated styrenics, and mixtures and combinations thereof.

Embodiment YY. The method of any of Embodiments QQ to XX, wherein the anti-biofouling layer comprises a material chosen from fluoropolymers, non-aromatic hydrocarbon polymers, cyclic olefin polymers, cyclic olefin copolymers, cyclic block copolymers, silicones, metals, a methyl terminated layer, a noble metal and mixtures and combinations thereof.

Embodiment ZZ. The method of any of Embodiments QQ to YY, wherein the inorganic layer comprises an oxide of Si, Ti or Al.

Embodiment AAA. The method of Embodiment ZZ, wherein the oxide is chosen from SiO₂, SiCₓO_{y}, SiAlₓO_{y}, TiO, Alₓ and mixtures and combinations thereof.

Embodiment BBB. The method of any of Embodiments QQ to AAA, wherein the analyte binding layer is chosen from a reactive silane, a functionizable hydrogel, a functionalizable polymer, and mixtures and combinations thereof.

Embodiment CCC. The method of Embodiment BBB, wherein the analyte binding layer comprises acrylamide copolymers, condensed silanes, and mixtures and combinations thereof.

Embodiment DDD. The method of any of Embodiments QQ to CCC, wherein the methylene groups are derived from a reaction with a functional silane.

Embodiment EEE. The method of Embodiment DDD, wherein the functional silane comprises functional groups chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, azos, and mixtures and combinations thereof.

Embodiment FFF. The method of Embodiment EEE, wherein the functional group is chosen from alkene, azide, ammo, carboxylic acid, hydrazone, halogen, hydroxy, tetrazoc, tetrazinc, thiol, norbornene and combinations thereof.

Embodiment GGG. The method of any of Embodiments: QQ to FFF, wherein the structures have a height above the first major surface of the flexible carrier film greater than 0 nm and less than 1000 nm.

Embodiment HHH. The method of any of Embodiments QQ to GGG, wherein the structures comprises posts with a diameter of 10 nm to 10000 nm.

Embodiment III. The method of any of Embodiments QQ to HRH, further comprising an adhesive layer on a second major surface of the flexible carrier film.

Embodiment JJJ. The method of Embodiment III, further comprising a support layer on the adhesive layer, wherein the support layer is chosen from a release liner and a rigid substrate chosen from silicon, glass, plastic, metal, metal oxide, paper, and combinations thereof.

Embodiment KKK. The method of any of Embodiments QQ to JJJ, wherein the analyte comprises a biomolecule chosen from amino acids, nucleosides, nucleotides, peptides, oligonucleotides, polynucleotides, nucleic acids, and proteins, carbohydrates, secondary metabolites, pharmaceutical molecules and combinations thereof.

Embodiment LLL. The method of Embodiment KKK, wherein the biomolecule is chosen from polynucleotides, oligonucleotides, and nucleic acids.

Embodiment MMM. A method for making a component of a diagnostic device, the method comprising:
applying an inorganic etch resist layer over a first major surface of a flexible carrier film;
overlaying an anti-biofouling layer on the inorganic etch resist layer;
providing a patterned layer with a patterned surface including a first arrangement of plateau features, each plateau feature adjoining at least one recessed feature, wherein the patterned layer is overlain by a masking layer;
contacting the anti-biofouling layer and the masking layer;
removing the patterned layer from the masking layer to transfer the masking layer to the anti-biofouling layer, the masking layer comprising an exposed surface with a second arrangement of recessed features and plateau features, wherein the second arrangement of recessed features in the masking layer is an inverse of the first arrangement of recessed features in the patterned layer;
etching the recessed features of the masking layer to remove the masking layer and a portion of the anti-biofouling layer to form in the recessed features in the second arrangement an array of wells configured to retain a sample fluid, wherein the wells have a floor comprising the inorganic layer and walls extending away from the floor, wherein the walls comprise a remainder portion of the anti-biofouling layer; and
selectively applying a functional silane material on the inorganic layers in the wells to form an analyte binding layer therein, wherein the analyte binding layer comprises at least one functional group reactive with an analyte in the sample fluid, and wherein the inorganic layers are bonded to the analyte binding layers with a network of methylene groups.

Embodiment NNN. The method of Embodiment MMM, wherein the etching comprises a fluorinated compound.

Embodiment OOO. The method of any of Embodiments MMM to NNN, further comprising applying a silane tie layer on the inorganic etch resist layer.

Embodiment PPP. The method of any of Embodiments MMM to OOO, further comprising forming a layer of nanostructures on the first major surface of the flexible carrier film.

Embodiment QQQ. The method of Embodiment PPP, wherein the nanostructures are formed on the first major surface of the flexible carrier film by plasma enhanced chemical vapor deposition (PECVD).

Embodiment RRR. The method of any of Embodiments MMM to QQQ, wherein the flexible carrier film comprises a polymeric material chosen from cyclic olefin polymer (COP), biaxially oriented polypropylene (BOPP), poly(meth)acrylates and copolymers, polyamides, polyesters, polycarbonates, hydrogenated styrenics, and mixtures and combinations thereof.

Embodiment SSS. The method of any of Embodiments MMM to RRR<, wherein the anti-biofouling layer comprises a fluoropolymer.

Embodiment TTT. The method of Embodiment SSS, further comprising applying a tie layer on the anti-biofouling layer.

Embodiment UUU. The method of any of Embodiments MMM to TTT, wherein the inorganic layer comprises an oxide of Si, Ti or Al.

Embodiment VVV. The method of Embodiment UUU, wherein the oxide is chosen from SiO₂, SiCₓO_{y}, SiAlₓO_{y}, TiO, AlOₓ and mixtures and combinations thereof.

Embodiment WWW. The method of any of Embodiments MMM to VVV, wherein the masking layer comprises a (meth)acrylate.

Embodiment XXX. The method of any of Embodiments MMM to WWW, wherein the analyte binding layer comprises acrylamides, silanes, and mixtures and combinations thereof.

Embodiment YYY. The method of Embodiment XXX, wherein the analyte binding layer comprises a silane chosen from an acrylate silane, an aminosilane, an alkoxy silane, and mixtures and combinations thereof.

Embodiment ZZZ. The method of Embodiment YYY, wherein the analyte binding layer comprises an alkoxysilane with an acrylamide functional group selected to form a hydrogel.

Embodiment AAAA. The method of Embodiment ZZZ, wherein the acrylamide is polymerizable to form a (poly)acrylamide in the wells.

Embodiment BBBB. The method of any of Embodiments MMM to AAAA, wherein the functional group in the analyte binding layer is chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, azos, and mixtures and combinations thereof.

Embodiment CCCC. Tb method of any of Embodiments MMM to BBBB, further applying an adhesive layer on a second major surface of the flexible carrier film.

Embodiment DDDD. The method of any of Embodiments MMM to CCCC, further comprising applying a support layer on the adhesive layer, wherein the support layer is chosen from a release liner and a rigid substrate chosen from silicon, glass, plastic, metal, metal oxide, paper, and combinations thereof.

Embodiment EEEE. The method of any of Embodiments MMM to DDDD, wherein the analyte comprises a biomolecule chosen from amino acids, nucleosides, nucleotides, peptides, oligonucleotides, polynucleotides, nucleic acids, and proteins, and combinations thereof.

Embodiment FFFF. The method of Embodiment EEEE, wherein the biomolecule is chosen from polynucleotides and nucleic acids.

Embodiment GGGG. The method of any of Embodiments MMM to FFFF, wherein the floors of at least some of the wells comprise surface structures.

Embodiment HHHH. The method of any of Embodiments MMM to GGGG, further comprising applying, prior to applying the inorganic masking layer to the first major surface of the flexible carrier film, a moldable layer to the first major surface of the flexible carrier film, and molding the moldable layer while in contact with the flexible carrier film to form an array of structures in the moldable layer.

Embodiment IIII. A method for making a component of a diagnostic device, the method comprising:
applying an inorganic etch resist layer over a first major surface of a flexible carrier film;
overlaying an anti-biofouling layer on at least a portion of the inorganic etch resist layer;
optionally overlaying a tie layer on the anti-biofouling layer;
providing a patterned layer with a patterned surface including a first arrangement of plateau features, each plateau feature adjoining at least one recessed feature, wherein the patterned surface is overlain with a masking layer;
overlaying the anti-biofouling layer on the masking layer;
removing the patterned layer from the masking layer to transfer the masking layer to the anti-biofouling layer, the masking layer comprising an exposed surface with a second arrangement of recessed features and plateau features, wherein the second arrangement of recessed features in the masking layer is an inverse of the first arrangement of recessed features in the patterned layer;
etching the recessed features of the mask layer with an etch material to remove a portion of the masking layer and a portion of the anti-biofouling layer to form in the recessed features of the second arrangement an array of wells configured to retain a sample fluid, wherein the wells have a floor comprising the inorganic etch resist layer and walls extending away from the floor, wherein the walls comprise a remainder portion of the anti-biofouling layer; applying, following the etching step, a peelable layer over the mask layer;
removing the carrier film and the peelable layer to remove the mask layer and the tie layer; and
selectively applying a functional silane material on the inorganic etch resist layers in the wells to form an analyte binding layer therein, wherein the analyte binding layer comprises at least one functional group reactive with an analyte in the sample fluid, and
wherein the inorganic layer is bonded to the analyte binding layer via a network of methylene linking groups.

Embodiment JJJJ. The method of Embodiment IIII, wherein the peelable layer comprises a material chosen from an adhesive, an acrylate, and mixtures and combinations thereof.

Embodiment KKKK. The method any of Embodiments IIII to JJJJJ, wherein the peelable layer comprises a mixture of poly(vinyl alcohol) (PVA) and a surfactant.

Embodiment LLLL. The method of any of Embodiments IIII to KKKK, wherein the peelable layer is deposited onto the patterned mold by mixing the peelable material with a solvent and subsequently evaporating the solvent.

Embodiment MMMM. The method of any of Embodiments IIII to LLLL, wherein a tie layer is added after the anti-biofouling layer.

Embodiment NNNN. The method of any Embodiments IIII to MMMM, wherein the tie layer has a thickness of about 10 nm.

Embodiment OOOO. The method of any of Embodiments IIII to NNNN, wherein the tie layer has a thickness of about 10 nm.

Embodiment PPPP. The method of any of Embodiments IIII to OOOO, wherein the tie layer comprises a poly(allyl) amine.

Embodiment QQQQ. The method of any of Embodiments IIII to PPPP, wherein the tie layer comprises a water soluble material with a thickness of about 10 nm to about 300 nm.

Embodiment RRRR. The method of any of Embodiments IIII to QQQQ, wherein the tie layer comprises poly(vinyl alcohol) (PVA).

Embodiment SSSS. The method of any of Embodiments IIII to RRRR, further comprising a second tie layer between the inorganic etch resist layer and the anti-biofouling layer.

Embodiment TTTT. The method of any embodiments IIII to SSSS, wherein the inorganic etch resist is structured or roughened on the nanoscale.

Embodiment UUUU. The method of any embodiments IIII to TTTT, wherein the functional silane material is applied to the inorganic etch resist layer after the etching step and before the mask layer is removed.

Embodiment VVVV. The method of any embodiments IIII to UUUU, wherein the functional silane material is applied to the inorganic etch resist after the mask layer is removed.

Embodiment WWWW. A diagnostic device for detection of a biochemical analyte, the diagnostic device comprising a flow cell with a patterned arrangement of fluidic channels configured to provide flow conduits for a sample fluid comprising the biochemical analyte, wherein at least some of the fluidic channels of the flow cell are lined on a surface thereof with:
an arrangement of posts comprising an analyte binding layer on an exposed surface thereof, or
an arrangement of wells comprising an analyte binding layer therein,
wherein the analyte binding layer is configured to bind the biochemical analyte, and wherein the analyte binding layer is bonded to an underlying Si oxide layer by a network of methylene groups disposed on a flexible carrier film.

Embodiment XXXX. The diagnostic device of Embodiment WWWW, wherein the flow cell surface is on a supporting substrate chosen from glass, plastic, silicon, metal, metal oxide, paper or a combination thereof.

Embodiment YYYY. A method for DNA sequencing, the method comprising:
in a diagnostic device comprising a flow cell with a patterned arrangement of fluidic channels configured to provide flow conduits for a sample fluid comprising a target analyte comprising polynucleotides and nucleic acids, wherein at least some of the fluidic channels of the flow cell are lined on a surface thereof with:
an arrangement of posts comprising an analyte binding layer on an exposed surface thereof, or
an arrangement of wells comprising an analyte binding layer therein,
wherein the analyte binding layer is configured to bind the biochemical analyte, and wherein the analyte binding layer is bonded to an underlying Si oxide layer by a network of methylene groups on a flexible carrier film;
binding the target analyte in the sample fluid on the analyte binding layer;
exposing the target analyte bound on the analyte binding layer to a fluorescent reagent and an enzyme such that the analyte is detected using spectroscopy; and
cleaving the fluorescent reagent to allow further interrogation of the target analyte.

Embodiment ZZZZ. The method of Embodiment YYYY, further comprising clonally amplifying the target analyte.

Embodiment AAAAA. The method of any of Embodiments YYYY to ZZZZ, wherein the fluorescent assay is sequencing by synthesis, combinatorial probe anchor synthesis, sequencing by ligation, single molecule real time sequencing, pyrosequencing, and combinations thereof.

Embodiment BBBBB. A DNA sequencing kit, comprising:
a diagnostic device comprising a flow cell with a patterned arrangement of fluidic channels configured to provide flow conduits for a sample fluid comprising a target analyte comprising polynucleotides and nucleic acids, wherein at least some of the fluidic channels of the flow cell are lined on a surface thereof with:
an arrangement of posts comprising an analyte binding layer on an exposed surface thereof, or
an arrangement of wells comprising an analyte binding layer therein,
wherein the analyte binding layer is configured to bind the biochemical analyte, and wherein the analyte binding layer is bonded to an underlying Si oxide layer by a network of methylene groups on a flexible carrier film;
fluorescent reagents for DNA sequencing; and
instructions.

Various embodiments of the invention have been described. These and other embodiments are within the scope of the following claims.

1. An article, comprising:
a flexible carrier film with a first major surface and a second major surface, wherein a first major surface of the flexible carrier film comprises an array of structures extending away therefrom, wherein at least a portion of the structures comprise:
an inorganic layer with a first major surface and a second major surface, wherein the first major surface of the inorganic layer is on the flexible carrier film,
an analyte binding layer with a first major surface on the second major surface of the inorganic layer, wherein the analyte binding is bonded to the inorganic layer via a network of hydrocarbon linking groups, and wherein the second major surface of the analyte binding layer comprises at least one functional group selected to bind with a biochemical analyte; and
recessed features interspersed with the structures, wherein at least a portion of the recessed features are free of the inorganic layer and the analyte binding layer.

2. The article of claim 1, wherein the flexible carrier film further comprises an anti-biofouling layer.

3. The article of claim 2, wherein the anti-biofouling layer comprises a material chosen from the group consisting of fluoropolymers, non-aromatic hydrocarbon polymers, cyclic olefin polymers, cyclic olefin copolymers, cyclic block copolymers, silicones, metals, a methyl terminated layer, a noble metal and mixtures and combinations thereof.

4. The article of claim 1, wherein the inorganic layer has a thickness of less than 100 nm.

5. The article of claim 1, wherein the inorganic layer comprises an oxide of silicon, titanium or aluminum.

6. The article of claim 1, wherein the analyte binding layer is chosen from the group consisting of a reactive silane, a functionalizable hydrogel, a functionalizable polymer, and mixtures and combinations thereof.

7. The article of claim 6, wherein the analyte binding layer comprises at least one of acrylamide copolymers and condensed silanes.

8. The article of claim 1, wherein the hydrocarbon linking group is at least one methylene unit long, and wherein the hydrocarbon linking group and can be linear, cyclic, branched, or aromatic and can optionally contain heteroatoms.

9. The article of claim 8, wherein the hydrocarbon linking group is derived from the condensation of a functional silane onto the inorganic layer, wherein the functional silane comprises functional groups chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, azos, and mixtures and combinations thereof.

10. The article of claim 1, wherein the structures comprise posts with a diameter of 10 nm to 10,000 nm.

11. The article of claim 1, further comprising an adhesive layer on the second major surface of the flexible carrier film.

12. The article of claim 11, further comprising a support layer on the adhesive layer, wherein the support layer is chosen from the group consisting of a release liner and a rigid substrate.

13. An article, comprising:
a flexible carrier film with a first major surface and a second major surface;
an inorganic layer with a first major surface and a second major surface, wherein the first major surface of the inorganic layer is on the first major surface of the flexible carrier film;
an anti-biofouling layer on at least a portion of the inorganic layer, wherein the anti-biofouling layer comprises an arrangement of wells, wherein at least a portion of the wells comprise a floor having thereon a first major surface of an analyte binding layer bound to the second major surface of the inorganic layer via a network of hydrocarbon linking groups, and wherein a first major surface of the analyte binding layer in the well comprises at least one functional group reactive with an analyte and
structures interspersed with the wells, wherein at least a portion of the structures are free of the analyte binding layer and the inorganic layer.

14. The article of claim 13, wherein the anti-biofouling layer comprises a material chosen from the group consisting of fluoropolymers, non-aromatic hydrocarbon polymers, cyclic olefin polymers, cyclic olefin copolymers, cyclic block copolymers, silicones, metals, a methyl terminated layer, a noble metal and mixtures and combinations thereof.

15. The article of claim 13, wherein the inorganic layer comprises an oxide of silicon, titanium or aluminum.

16. The article of claim 13, wherein the analyte binding layer is chosen from the group consisting of silane, a functionalizable hydrogel, a functionalizable polymer, and mixtures and combinations thereof.

17. The article of claim 13, wherein the hydrocarbon linking group is derived from a reaction with a functional silane having functional groups chosen from epoxides, oxiranes, aziridines, isocyanates, alcohols, thiols, amines, chloromethylbenzyl, bromomethylbenzyl, iodomethylbenzyl, alkyl halides, vinyl, carbonyls such as aldehydes and ketones, carboxylic acids, esters, azides, sulfates, phosphates, alkenes, alkynes, (meth)acrylates, (meth)acrylamides, norbornenes, diazonium salts, hydrazines, hydrazones, oximes, halogens, hydroxyls, tetrazoles, tetrazines, benzophenones, aryl azides, halogenated aryl azides, diazos, azos, and mixtures and combinations thereof.

18. The article of claim 13, wherein the wells have a diameter of 10 nm to 10,000 nm.

19. The article of claim 13, wherein the inorganic layer has a thickness of less than 100 nm.

20. A diagnostic device for detection of a biochemical analyte, the diagnostic device comprising a flow cell with a patterned arrangement of fluidic channels configured to provide flow conduits for a sample fluid comprising the biochemical analyte, wherein at least some of the fluidic channels of the flow cell are lined on a surface thereof with:
an arrangement of posts comprising an analyte binding layer on an exposed surface thereof, or
an arrangement of wells comprising an analyte binding layer therein, wherein the analyte binding layer is configured to bind the biochemical analyte, and wherein the analyte binding layer is bonded to an underlying silicon oxide layer by a network of methylene groups disposed on a flexible carrier film.
